(19) United States
(12) Patent Application Publication
(10) Pub. No.: US 2025/0164503 A1
(43) Pub. Date: May 22, 2025

(54) ENGINEERED RECOMBINANT PROTEIN-BINDING DOMAINS AS DETECTION REAGENTS

(71) Applicant: EPICYPHER, INC., Durham, NC (US)

(72) Inventors: Martis W. Cowles, Chapel Hill, NC (US); Zu-Wen Sun, Brentwood, TN (US); Bryan Venters, Apex, NC (US); Matthew Marunde, Carrboro, NC (US); Jonathan Burg, Durham, NC (US); Michael-Christopher Keogh, Cambridge, MA (US)

(21) Appl. No.: 18/838,404

(22) PCT Filed: Feb. 15, 2023
(86) PCT No.: PCT/US2023/062629

§ 371 Date: Aug. 14, 2024

Related U.S. Application Data
(60) Provisional application No. 63/310,241, filed on Feb. 15, 2022.

(51) Int. Cl. G01N 33/68; C07K 14/47

(52) U.S. Cl. CPC G01N 33/6875; C07K 14/47; C07K 2319/23; C07K 2319/24

(57) ABSTRACT

Disclosed herein are methods to assay and identify detection reagents for chromatin elements, including recombinant fusion protein detection reagents comprising two or more binding domains for chromatin elements. Improved genomic mapping assays using the identified recombinant proteins are also disclosed. Recombinant fusion proteins optimized by the disclosed methods are also provided.

### STATEMENT OF PRIORITY

This application claims the benefit of U.S. Provisional Application Ser. No. 63/310,241, filed Feb. 15, 2022, the entire contents of which are incorporated by reference herein.

### FIELD OF INVENTION

The invention relates to improved genomic mapping assays, recombinant proteins and compositions for use in the assays.

### STATEMENT REGARDING ELECTRONIC FILING OF A SEQUENCE LISTING

A Sequence Listing in XML format, entitled 1426-41WO_ST26.xml, 61,440 bytes in size, generated on Feb. 15, 2023 and filed herewith, is hereby incorporated by reference in its entirety for its disclosures.

### BACKGROUND

Nucleosomes are the repeating units of chromatin, consisting of 147 bp DNA wrapped around histone octamers. Histones are modified by the addition or removal of various PTMs (e.g., lysine methylation and acetylation), which alter the global chromatin environment to regulate fundamental cellular processes that include gene transcription and cell differentiation. Over 100 different histone PTMs have been identified, and individual PTMs are associated with differing downstream effects (e.g., H3K4me3 is associated with active transcription, whereas H3K27me3 is associated with repressed genes). Further, unmodified histone residues (i.e., the absence of specific PTMs) also contribute to the histone code and play important roles in DNA replication (e.g., H4K20me0 marks post-replicative chromatin and recruits DNA repair machinery) and other processes. Critically, dysregulation of the chromatin machinery that writes, erases, and interprets histone PTMs is associated with a range of human diseases, including neurodegeneration, metabolic syndrome, and cancer. Accordingly, recent exploration of histone PTMs (and downstream effectors) and their biological roles is driving clinical advances and discovery of new therapeutics and biomarkers. Many major pharmaceutical companies have initiated epigenetics programs to discover and develop novel drugs targeting PTMs, a number of which are in clinical trials and/or approved for clinical use.

The ability to accurately map histone PTMs, DNA modifications, and other chromatin-bound proteins in epigenomic assays and other applications is reliant upon high quality detection reagents, which must exhibit high on-target epitope binding (i.e., efficiency) with minimal off-target binding (i.e., specificity). The accuracy of antibodies is a serious and increasing concern in the biomedical field, and >$750 million (M) is spent worldwide each year on nonspecific or “bad” antibodies. This is particularly true for chromatin-targeting antibodies, which are plagued by poor specificity and low enrichment. Histone PTMs (and unmodified histone residues) are particularly challenging targets for antibody development as they are small in size, chemically similar to one another, and often co-occur with other modifications or surrounding motifs that influence their detection.

The majority of histone PTM antibodies are validated using peptides, which poorly mimic the three-dimensional structure of an intact nucleosome that is essential to recapitulate chromatin interactions.

To begin to address the challenges in characterizing chromatin targeting antibodies, the present inventors and others developed DNA-barcoded nucleosomes as spike-in controls for epigenomic mapping assays. These reagents are comprised of pools of DNA-barcoded designer nucleosomes (dNucs) carrying on- and related off-target epitopes (e.g., histone PTMs, or chromatin binding domain epitopes) as controls to directly assess antibody performance. The inventors recently used these DNA-barcoded dNuc spike-ins to characterize >400 commercial PTM antibodies. Strikingly, it was found that >70% display unacceptable off-target binding and/or low on-target recovery (i.e., low efficiency), making them unsuitable for use (chromatinantibodies.com). It has also been observed that antibody binding on histone peptides had minimal correlation with binding specificity in a nucleosome context (52 antibodies, R2=0.2337), highlighting the importance of the use of nucleosome substrates to characterize PTM affinity antibodies. While studies have found antibodies suitable for use for some marks, there is a significant number of PTMs (e.g., H4K20me2) with biological/disease relevance for which no high-quality antibodies exist, leaving these marks functionally inaccessible. This precludes these high-value marks from being effectively studied and developed as drug targets and/or potential biomarkers. Further, with the advent of single cell and spatial epigenomic mapping assays, there is a growing need for detection reagents with not only high specificity, but also binding efficiency.

Epigenomic modifications on chromatin, consisting of histone PTMs and DNA methylation, do not exist in isolation. Rather, the combination of multiple modifications orchestrates dynamic processes, creating a complex language known as the “histone code”. Proteins (or complexes) that bind chromatin (aka “readers”) often interact with specific combinations of histone PTMs, resulting in cooperative binding events that are stronger and more specific than interactions between single chromatin reader domains and their individual targets (e.g., TFIID transcription complex displays ˜8-fold increase in binding on poly-acetylated H4 vs. H4K16ac). These multivalent interactions with combinatorial PTMs are a key mechanism by which the histone code functions and mediates effects on the cellular environment. Indeed, specific combinations of PTMs have been associated with cellular processes and disease states and can be more informative and reliable than individual PTMs. For example, bivalent domains are marked by both active (e.g., H3K4me3) and repressive (e.g., H3K27me3) marks, creating a chromatin state that is poised for rapid transcriptional activation. Bivalent regions in ES cells are prone to aberrant DNA methylation in cancer, and disruption of bivalency is a common feature of multiple cancer cell lines. While cancer cells have fewer bivalent domains overall compared to normal cells, some cancer cell lines exhibit partial recapitulation of bivalent chromatin modifications. Thus, the study of combinatorial PTMs is highly relevant to our understanding of human disease, and may unlock new biomarkers and/or therapeutic targets for cancer and other diseases.

Existing reagents and strategies to detect and profile histone and/or DNA modifications are insufficient for interrogating co-incident modifications. For instance, mass spectrometry (MS) has historically been important in the study of dual PTMs; however, it is technically and analytically challenging, low throughput, and limited to modifications on the same histone tail or modifications that co-occur on histones and DNA. Furthermore, MS only provides relative amounts of single and dual PTMs, and is not amenable to genome-wide PTM mapping, which is important to better understand histone code function and discover novel biomarkers or drug targets. Current chromatin profiling studies largely rely on chromatin immunoprecipitation sequencing (ChIP-seq), which is useful in mapping single PTMs, but is highly limited in its application to combinatorial PTMs. Given the lack of commercially available detection reagents for dual PTMs, they are typically profiled in parallel ChIP-seq assays (which reveal global co-enrichment but not specific co-localization) or sequentially in re-ChIP experiments (which require large inputs and are highly inefficient). Furthermore, ChIP methodologies are time-intensive, low-resolution, display low signal-to-noise (S/N), and are qualitative in nature (i.e., lack spike-in controls). The ability to reliably detect combinatorial PTMs (and study their genome-wide distribution) would facilitate a deeper understanding of the role of dual PTMs in disease, and may reveal new diagnostic indicators with increased specificity compared to single PTMs or bulk co-enrichment measurements.

Recombinant domains have been previously used in isolation or linked with other protein domains as detection reagents. While these approaches hold great promise, it remains difficult to characterize/optimize the recombinant protein to have optimal binding on chromatin, which is comprised of DNA wrapped around histone proteins to make nucleosomes. Indeed, current assays lack the ability to faithfully monitor the specificity of recombinant protein domains as detection reagents for chromatin studies.

### INVENTION SUMMARY

The present invention is based, in part, on development of novel detection reagents that use two or more protein domains to map two or more modifications on a single nucleosome or adjacent nucleosomes and development of novel detection reagents that fuse together two or more of the protein domains to improve assay sensitivity. The novel detection reagents are used for improved sensitivity and selectivity in chromatin assays (e.g., genomics assays or binding assays), including genomic mapping assays such as CUT&RUN and CUT&Tag.

In an aspect, a method for screening a screening a detection reagent for a chromatin element is provided, comprising: providing a panel of recombinant nucleosomes, each nucleosome comprising one or more chromatin elements recognized by the one or more binding domains present in the detection reagent and/or one or more chromatin elements not recognized by the one or more binding domains present in the detection reagent, thereby providing both on-target and off-target recombinant nucleosomes; and performing a genomics assay or binding assay with the recombinant fusion protein detection reagent comprised of one or more binding domains that recognize one or more of the chromatin elements of at least one of the nucleosomes in the panel and a label, and the panel of recombinant nucleosomes to identify the binding specificity and/or efficiency of the detection reagent. In an embodiment, the detection reagent comprises a recombinant fusion protein comprising one or more binding domains that recognize one or more of the chromatin elements in the panel of nucleosomes, and a label.

In another aspect, genomic mapping assay methods are provided, wherein the detection reagent is comprised of a fusion protein that contains one or more binding domains, wherein when two or more binding domains are present, they can optionally joined together by a linker, and a label, e.g., an epitope tag.

In another aspect, recombinant fusion proteins comprise two or more binding domains which may be selected from bromodomains, chromodomains, PWWP domains, plant homeodomains (PHD), malignant brain tumor (MBT) domain, WD40 Repeat domain, Tandem Tudor and/or Tudor-like domains. The recombinant fusion proteins can be designed and validated utilizing the methods described herein. In an aspect, the recombinant fusion proteins can comprise two or more of the same binding domains, or functional fragments thereof, and the binding efficiency and/or binding specificity of the fusion protein in genomic assays can be increased by at least 5-fold for the chromatin element recognized by the binding domains relative to an off-target chromatin element. In another aspect, recombinant fusion proteins can be designed and validated utilizing the methods described herein. In an aspect, the recombinant fusion proteins can comprise two or more different binding domains, or functional fragments thereof, each domain recognizing one or more chromatin elements. The fusion protein can be capable of specifically binding to two or more chromatin targets with an increased binding efficiency and/or binding specificity of at least 5-fold relative to a single chromatin target.

In an aspect, kits for carrying out the methods of the present invention and/or comprising the detection reagent are also provided.

These and other aspects of the invention are set forth in more detail in the description of the invention below.

### BRIEF DESCRIPTION OF THE DRAWINGS

FIGS. 1A-1B show engineered recombinant proteins as detection reagents for chromatin studies. (1A) Super Readers are novel engineered super-affinity reagents that contain multiple domains from the same chromatin binding protein. (1B) Cooperative multivalent binding of Tandem Readers, also referred to as an EpiTandem Sensor, on combinatorially-modified dNucs (vs. single PTMs); no binding on unmodified dNucs (or off-target PTMs).

FIGS. 2A-2B show dCypher assay schematics using modified nucleosome substrates. (2A) Alpha dCypher assay schematic, a bead-based proximity (singleplex) assay. In an exemplary workflow, biotinylated nucleosomes are incubated in the presence of recombinant fusion reader protein (e.g., Super Reader or Tandem Reader) containing an epitope tag. After (or during) the binding reaction the following exemplary reagents are added to reaction mixture; 1) streptavidin coated donor beads that interact with the recombinant nucleosome, 2) an antibody that binds to the epitope tag of the reader fusion protein, and 3) an acceptor bead that binds the antibody that is bound to the reader fusion protein. Binding of the recombinant fusion protein (e.g., Super Reader or Tandem Reader) is quantified by detecting the amount of acceptor bead emission, which only occurs when the nucleosome and recombinant fusion protein are bound. (2B) Luminex dCypher schematic, an exemplary bead-based multiplex assay. In this example, one or more biotinylated nucleosomes carrying different histone PTMs or chromatin associated protein (CAP) epitopes are conjugated to xMAP streptavidin coated magnetic beads (Step 1). Next, nucleosome-bound beads are incubated with a recombinant fusion protein carrying an epitope tag (Step 2). Next, a primary antibody that contains a fluorophore is bound to the epitope tag; alternatively, a secondary antibody can be bound to the primary antibody and the fluorophore is on the first and/or second antibody (Step 3). Finally, the reaction mix is quantified on a Luminex plate reader (Step 4).

FIGS. 3A-3C show dCypher® assay profiling of the (3A) 1×CHD of CBX1 (3B) 2×CHD of CBX1 and (3C) 3×CHD of CBX1 H3K9me3 Super Reader construct. Super Reader H3K9me3 constructs contained either single or multiple copies of CHD of CBX1 in series with an N-terminal MBP tag. These constructs were then subjected to dCypher® profiling, as detailed in the working examples. Although the 1×CBX1 CHD construct maintains approximately a 10-fold relative binding specificity toward H3K9me3 over H3K9me2, the 3×CBX CHD construct improves this to greater than 100-fold with minimal change in off-target binding. This experiment suggests that the avidity gain and multivalency of domain multimerization is an effective step toward generating high affinity reagents.

FIG. 4 includes a graphical depiction of a DNA-barcoded dNuc spike-in panel for profiling the H3K9me3 Super Reader in a modified CUT&RUN workflow.

FIG. 5 includes a graphical representation of Super Reader CUT&RUN workflow.

FIG. 6 shows H3K9me3 mapped with antibody in mouse B cells. In this CUT&RUN experiment, a best-in-class H3K9me3 antibody was utilized. Of note, the H3K9me3 genomic locations are distinct from H3K27me3, which are a known marker of heterochromatic regions.

FIGS. 7A-7B show CUT&RUN with H3K9me3 Super Reader showing (7A) total DNA yields prior to library preparation and (7B) validation of specificity with dNuc spike-in controls, showing high on-target recovery with multimerized constructs. Data is expressed for each spike-in dNuc barcode as a percent of the total number of barcode reads. These results show that the 3×CBX1 construct recovers an indistinguishable amount of DNA as compared to the best-in class H3K9me3 antibody. Additionally, the 3×CBX1 construct maintains >10-fold preference for its preferred target (H3K9me3).

FIG. 8 shows genome browser tracks of CUT&RUN data, using the H3K9me3 Super Reader (3×CBX1), 1× and 2×CBX1 constructs, H3K9me3 antibody, and relevant controls. Note that for the 3×CBX1 Super Reader construct, the peak structure and genome-wide mapping of the best-in-class H3K9me3 antibody was faithfully reproduced.

FIG. 9 shows genome browser tracks of CUT&RUN data, using the H3K4me3 Super Reader 1×, 2×, and 3×TAF3 PHD constructs, H3K4me3 antibody, and relevant controls. Of note, using the 1×TAF3 PHD Super Reader construct the peak structure and genome-wide distribution of the best-in-class H3K4me3 antibody was faithfully reproduced.

FIGS. 10A-10D show profiling of BPTF Tandem Reader sensor specificity using dCypher®-Alpha. (10A, 10C) BPTF Tandem Reader sensor was used to probe a library of (10A) dNuc or (10C) histone peptides. (10B, 10D) The BPTF Tandem Reader sensor was titrated against top interactors from (10A) on singly- and combinatorially-modified nucleosomes, plus unmodified (10B) dNucs or (10D) histone peptides. Importantly, a gain of relative affinity toward a combinatorially modified substrate was only noted on nucleosomal targets suggesting that there are additional determinates of specificity in a nucleosomal context that cannot be denoted on peptide targets.

FIGS. 11A-11C show combinatorial dNuc quality validation. (11A) ESI-TOF MS of H3K4me3+K9,14,18ac peptide (expected mass=3661.23 Da/observed mass=3661.13 Da). (11B) Agarose gel showing no contaminating free-DNA. (11C) Octamer validation illustrating the presence of equal molar amounts of each of the four histones in the nucleosome octamer (Coomassie staining).

FIGS. 12A-12D show profiling of binding specificity of artificial Sensors using dCypher®-Luminex®. Tandem Reader Sensors were tested against (12A) methyl-lysine dNuc set, (12B) single and combinatorial dNucs, then (12C) titrated against single and combinatorial dNucs. (12D) The Tandem Reader Sensor without a linker displayed the strongest dual binding activity compared to the Sensors with linkers. All linker designs detailed in Example 5 were tested in (12A-12C), but based on (12D), only results from the Tandem Reader Sensor with no linker are shown.

FIG. 13 shows a graphical representation of the CUT&RUN dNuc spike-ins for profiling the specificity of the BPTF Tandem Reader Sensor in a CUT&RUN workflow.

FIG. 14 is a graphical representation of CUT&RUN workflow using both Tandem Reader Sensors and DNA barcoded combinatorially-modified dNuc spike-ins.

FIG. 15 shows validation of BPTF Tandem Reader Sensor with dNuc spike-in controls. Data is expressed for each spike-in dNuc barcode as a percent of the total number of barcode reads. Importantly, the BPTF Tandem Reader Sensor shows an 8-fold preference toward the H3K4me3+K9,14,18ac containing combinatorially modified nucleosome over nucleosome containing only a single modification.

FIG. 16 shows CUT&RUN signal distribution at promoters. BPTF Sensor recapitulates localization of endogenous BPTF; however, the distribution around TSS is broadened, similar to H3K4me3 and H3K9ac profiles.

FIGS. 17A-17B show genome browser (IGV) tracks of CUT&RUN data, using the BPTF Tandem Reader Sensor and controls. (17A) Both endogenous BPTF and the GST-tagged BPTF Tandem Reader Sensor only bind where there is a co-occurrence of both H3 acetylation and H3K4me3. (17B) The BPTF Tandem Reader Sensor binds additional H3K4me3+H3K-acetyl sites vs. endogenous BPTF, thus providing a comprehensive view of dual PTM enrichment, but also suggesting there is secondary regulation of endogenously expressed full-length BPTF.

### DETAILED DESCRIPTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art. In addition, any references cited herein are incorporated by reference in their entireties.

Unless otherwise defined, all technical and scientific terms used herein have the same meaning as commonly understood by one of skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Amino acids are represented herein in the manner recommended by the IUPAC-IUB Biochemical Nomenclature Commission, or (for amino acids) by either the one-letter code, or the three-letter code, both in accordance with 37 C.F.R. § 1.822 and established usage.

Except as otherwise indicated, standard methods known to those skilled in the art may be used for cloning genes, amplifying, and detecting nucleic acids, and the like. Such techniques are known to those skilled in the art. See, e.g., Sambrook et al., Molecular Cloning: A Laboratory Manual 4th Ed. (Cold Spring Harbor, NY, 2012); Ausubel et al. Current Protocols in Molecular Biology (Green Publishing Associates, Inc. and John Wiley & Sons, Inc., New York).

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a complex comprises components A, B and C, it is specifically intended that any of A, B or C, or a combination thereof, can be omitted and disclaimed singularly or in any combination.

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±10%, ±5%, ±1%, ±0.5%, or even ±0.1% of the specified amount.

As used herein, the transitional phrase “consisting essentially of” (and grammatical variants) is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “consists essentially of” (and grammatical variants), as applied to a polypeptide or polynucleotide sequence of this invention, means a polypeptide or polynucleotide that consists of both the recited sequence (e.g., SEQ ID NO) and a total of ten or less (e.g., 1, 2, 3, 4, 5, 6, 7, 8, 9, or 10) additional amino acids on the N-terminal and/or C-terminal ends of the recited sequence or additional nucleotides on the 5′ and/or 3′ ends of the recited sequence such that the function of the polypeptide or polynucleotide is not materially altered. The total of ten or less additional amino acids or nucleotides includes the total number of additional amino acids or nucleotides on both ends added together. The term “materially altered,” as applied to polypeptides of the invention, refers to an increase or decrease in biological activities/properties (e.g., remodeling activity) of at least about 50% or more as compared to the activity of a polypeptide consisting of the recited sequence.

As used herein, the term “polypeptide” encompasses both peptides and proteins, unless indicated otherwise.

The terms “polynucleotide,” “nucleic acid,” “nucleic acid molecule,” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, genomic DNA, chimeras of RNA and DNA, isolated DNA of any sequence, isolated RNA of any sequence, synthetic DNA of any sequence (e.g., chemically synthesized), synthetic RNA of any sequence (e.g., chemically synthesized), nucleic acid probes and primers. A polynucleotide can comprise modified nucleotides, such as methylated nucleotides and nucleotide analogs or derivatives (e.g., inosine or phosphorothioate nucleotides). Such nucleotides can be used, for example, to prepare nucleic acid molecules that have altered base-pairing abilities or increased resistance to nucleases.

As used herein, a “functional” polypeptide or “functional fragment” is one that substantially retains at least one biological activity normally associated with that polypeptide (e.g., wild-type protein or fragment thereof). In particular embodiments, the “functional” polypeptide or “functional fragment” substantially retains all of the activities possessed by the unmodified polypeptide (e.g., wild-type protein or fragment thereof). By “substantially retains” biological activity, it is meant that the polypeptide retains at least about 20%, 30%, 40%, 50%, 60%, 75%, 85%, 90%, 95%, 97%, 98%, 99%, or more, of the biological activity of the native polypeptide (and can even have a higher level of activity than the native polypeptide). A “non-functional” polypeptide is one that exhibits little or essentially no detectable biological activity normally associated with the polypeptide (e.g., at most, only an insignificant amount, e.g., less than about 10% or even 5%). Biological activities such as chromatin binding activity can be measured using assays that are well known in the art and as described herein.

The term “fragment,” as applied to a peptide, will be understood to mean an amino acid sequence of reduced length relative to a reference peptide (e.g., wild-type protein) or amino acid sequence and comprising, consisting essentially of, and/or consisting of an amino acid sequence of contiguous amino acids identical to the reference peptide or amino acid sequence. Such a peptide fragment according to the invention may be, where appropriate, included in a larger polypeptide of which it is a constituent. In some embodiments, such fragments can comprise, consist essentially of, and/or consist of peptides having a length of at least about 5, 10, 15, 20, 25, 30, 35, 46, 50, 55, 60, 65, 70, 75, 80, 85, 90, 95 or 100 or more consecutive amino acids of a peptide or amino acid sequence according to the invention.

The term “modulate,” “modulates,” or “modulation” refers to enhancement (e.g., an increase) or inhibition (e.g., a decrease) in the specified level or activity.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold and/or can be expressed in the enhancement and/or increase of a specified level and/or activity of at least about 1%, 5%, 10%, 15%, 25%, 35%, 40%, 50%, 60%, 75%, 80%, 90%, 95% or more.

The term “inhibit” or “reduce” or grammatical variations thereof as used herein refers to a decrease or diminishment in the specified level or activity of at least about 1, 5, 10, 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

The term “contact” or grammatical variations thereof refers to bringing two or more substances in sufficiently close proximity to each other for one to exert a biological effect on the other.

The present invention relates to improved chromatin assay methods. The improved chromatin assay methods comprise optimized or improved detection reagents for a chromatin element. In some embodiments, the present invention relates to methods for designing and validating r detection reagents for chromatin targets, such as transcription factors, histone post-translational modifications, and modified DNA. The present invention describes methods that use modified recombinant nucleosome substrates to systematically evaluate how changes in the recombinant protein design and reaction conditions (e.g. concentration, buffers, etc.) alter the specificity and binding efficiency of these targets in vitro and in vivo. The invention further relates to methods that use said engineered domains in chromatin assays as well as assay kits that include the reagents needed to perform various chromatin assays.

In an embodiment, the present invention relates to biochemical and genomics assays that use engineered recombinant fusion proteins, e.g., Super Reader or Tandem Reader proteins. Example assays include be any assay that uses antibody reagents as detection reagents, such as immunoassays, immunoblots, chromatin mapping, immunocytochemistry, immunohistochemistry, and immunofluorescent assays. In an embodiment, the assay is a genomics assay, which includes assays for identification, comparison, or measurement of genomic features including DNA sequence, structural variation, gene expression, and gene function, and including chromatin assays. Example chromatin assays that use the recombinant fusion proteins of the present invention include ChIP-seq, Cleavage under targets and release using nuclease (CUT&RUN) (Skene et al., (2017) An efficient targeted nuclease strategy for high-resolution mapping of DNA binding sites eLife 6: e21856; doi: 10.7554/eLife.21856), Cleavage under targets and tagmentation (CUT&Tag) (Kaya-Okur, et al. CUT&Tag for efficient epigenomic profiling of small samples and single cells. Nat Commun 10, 1930 (2019); doi: 10.1038/s41467-019-09982-5) or any other genomics assay known in the art including those as described in International Patent Publication Nos. WO 2019/060907 and WO 2018/042251, incorporated herein by reference in their entirety. Samples for chromatin assays include cells, nuclei, or biological fluids (e.g., cell-free nucleosomes). Biological samples for genomic assays performed on biological fluids, can include, for example, samples of blood, plasma, saliva, stool, and spinal fluid. In an aspect, the genomic mapping assay is performed on <10,000 cells, <1,000-2,000 cells, or on a single cell.

In an embodiment, the improved detection reagent is a recombinant fusion protein, which may use one or more recombinant proteins, functional fragments or domains thereof linked together in a recombinant fusion protein that binds one or more chromatin element (e.g., histone post-translational modification, DNA modification, histone mutation, histone variant, and/or proteins that indirectly or directly bind chromatin). These recombinant fusion proteins are used as detection reagents to perform chromatin mapping assays, allowing one or more chromatin element to be measured on a single nucleosome or adjacent nucleosomes and/or providing improved sensitivity for low cell input assays (vs. current best-in-class antibodies). The recombinant fusion proteins have been optimized in assays that show that an isolated histone binding domain can exhibit different binding properties (e.g., specificity, efficiency) when targeting a histone peptide versus a nucleosome, which is surprising and not yet appreciated in the art. The present invention leverages this novel finding to enable the systematic development of next-generation detection reagents for chromatin studies. The recombinant fusion proteins can be optimized in assays with additional reagents of the assay to find the combination of fusion protein and detection reagents including, for example, spike-in nucleosome panels, controls, buffers, and enzymes that work best together in the genomic assay for increased specificity and efficiency.

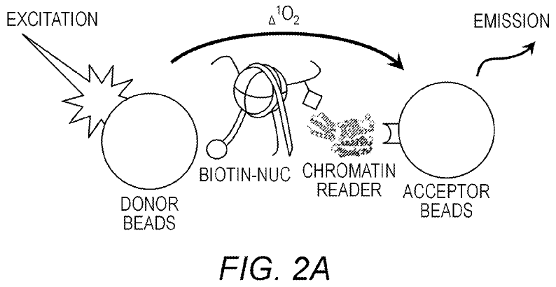

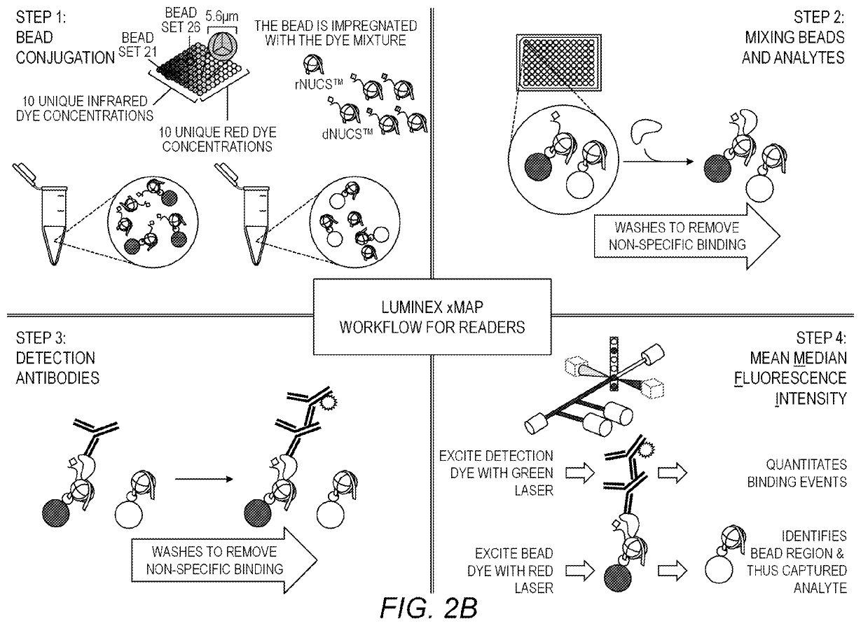

In some embodiments, engineered reader proteins, e.g., recombinant fusion proteins, are used as detection reagents in high-throughput dCypher® assays (see FIG. 2A-2B) to profile the binding activity of protein (e.g., reader or binder) domains against a panel of DNA-barcoded designer nucleosomes (dNucs) carrying a diverse array of PTMs. In some embodiments the assay is a binding assay, which is any assay that measures interactions between two molecules, in this instance, an assay that measures the binding of a recombinant fusion protein. In an embodiment, the binding assay may be, for example, a bead-based proximity assay or bead-based multiplex assay bead based assay, wherein detection reagents (e.g., antibodies or chromatin readers) are profiled against a panel of dNucs coupled to spectrally barcoded Luminex xMAP beads for rapid evaluation of binding activity to multiple targets in a single reaction (FIG. 2B). The emitted Luminex signal for each barcode is proportional to the degree of interaction between the dNuc and the detection reagent. Example steps in the assay comprise providing sets beads each impregnated with a unique dye (e.g., infrared dye) and conjugating the sets of beads to dNucs and mixing beads and analytes, including the detection reagent being screened. One or more washes can be performed to remove any non-specific binding. Detection antibodies are utilized to specifically bind to labeled detection reagent bound to bead functionalized dNucs. One or more washes can be performed again at this stage to remove any non-specific binding. Measuring median fluorescence intensity can then be performed using a wavelength specific to excite the detection antibody to quantitate binding events and a wavelength specific for the unique bead dye, identifying the bead region and thus the captured analyte, i.e., detection reagent. Notably, dCypher® screening is amenable to testing with various wash buffers to optimize and predict binding activity in specific downstream assays (e.g., ELISA, CUT&RUN, CUT&Tag, ChIP-seq, etc.). In some embodiments, the protein design and assay conditions optimized using the dCypher® assay are used to develop optimal conditions for genomic mapping assays, such as CUT&RUN, CUT&Tag, and/or ChIP-seq.

Example genomic mapping assays useful with the invention include CUT&RUN, CUT&Tag, and ChIP-seq. Genomic mapping assays can comprise the steps of capturing cells on a solid support, permeabilizing the cells and incubating the cells with the labeled detection reagent for a chromatin element, excising the nucleosomes, extracting and analyzing to thereby identify chromatin element binding. For ChIP seq, steps include crosslinking the DNA and the protein in live cells, lysing the cells and extracting and shearing the chromatin, immunoprecipitating with an antibody targeting the protein of interest, followed by extracting the DNA from the protein. Evaluating the chromatin elements can be performed at specific regions of interest within the genome by quantitative PCR (qPCR) or genome-wide by Next Generation Sequencing.

In embodiments, DNA yields of the recombinant fusion proteins are higher than that for chromatin element-specific antibodies, for example, 2-fold, 3-fold, 4-fold, 5-fold, 6-fold, 7-fold, 8-fold, 9-fold, or 10-fold higher. In embodiments, the recombinant fusion proteins comprising two or more different protein domains display an increased binding preference for a combination of chromatin elements over single chromatin elements for which each protein domain is specific.

### Recombinant Fusion Proteins

The detection reagents useful in the improved genomic mapping assays comprise recombinant fusion proteins. The recombinant fusion proteins may comprise two or more binding domains, or functional fragments thereof, joined together with or without a linker. The recombinant protein can comprise one or more labels and one or more recognition sequences. The two or more binding domains may be the same or different. In some embodiments, recombinant fusion proteins are optimized using nucleosome substrates. In some embodiments, the recombinant fusion protein can be optimized for genomic mapping by using DNA-barcoded recombinant nucleosomes as spike-in controls to monitor binding specificity and/or efficiency in, for example, a genomics mapping assay workflow. In some embodiments, the recombinant fusion protein comprises a protein with 80%, 85%, 90%, 91%, 92%, 93%, 94%, 95%, 96%, 97%, 98%, 99% sequence identity to a protein in Table 1 (SEQ ID NOs.: 7-47). In some embodiments, the recombinant fusion protein is selected from the proteins in Table 1. In some embodiments, the recombinant fusion proteins comprise one or more domains with at least 80%, 85%, 90%, 91%, 92%, 93%, 94%, 95%, 96%, 97%, 98%, 99% sequence identity to the domains identified in Table 1. In some embodiments, the recombinant fusion proteins comprise one or more domains identified in Table 1.

In some embodiments, the binding of the fusion protein comprising two or more of the same binding domain (a super reader) is increased by at least 5-fold for a single chromatin element relative to other chromatin elements in an assay or off-target chromatin element.

The fusion proteins comprising two or more different binding domains (a tandem reader) that are designed and validated by the methods of the invention can be capable of specifically binding to two or more chromatin elements with an increased preference at least 5-fold relative to a single chromatin element.

In an embodiment, the improved detection reagents comprise two or more protein domains, or functional fragments thereof. The proteins, protein domains, or functional fragments thereof, are typically derived from proteins (or complexes) that bind chromatin (also referred to as “readers”). Binding domains, proteins, or functional fragments thereof useful for the fusion proteins of the present invention can be derived from a remodeling enzyme or remodeling enzyme complex. The term remodeling enzyme or remodeling complex refers to enzymes that interact with chromatin, in particular the DNA and/or histones in the chromatin. The remodeling enzyme may be an ATP-dependent chromatin remodeling complex, including, but not limited to, the imitation switch (ISWI) family, CHD, INO80, SWI/SNF and ATRX. Subfamilies of the ISWI family can be used, including ISWIA, IS1b, ISW2, NURF, CHRAC, ACF, WICH, RORC, RSF, CERF, and orthologs and homologs thereof. Accordingly, exemplary remodeling enzymes from which proteins, protein domains and functional fragments thereof can be derived include SWI/SNF, RSC, ISW1a (ATP-dependent) NuRD family, BPTF bromodomain PHD finger TF (see, e.g., Zahid et al., “Opportunity Knocks for Uncovering New Function of an Understudied Nucleosome Remodeling Complex Member, the Bromodomain PHD Finger Transcription Factor, BPTF” Curr Opin Chem Biol. 2021 August; 63:57-67; doi: 10.1016/j.cbpa.2021.02.003), PBAF, INO80, ATP-utilizing chromatin assembly and remodeling factor (ACF) complex, including drosophila ACF complex (dmACF), Human SMARCA2/BRM (SMARCA2), Human SMARCA4/BRG1 (SMARCA4), and human SMARCA5/SNF2H (SMARCA5).

Exemplary protein domains can include chromodomains, bromodomains, MBT domains (see, e.g., Bonasio et al., Semin Cell Dev Biol. 2010 April; 21 (2): 221-230; doi: 10.1016/j.semcdb.2009.09.010), plant homeodomains (PHD), WD40 Repeat domains (see, e.g., Schapira, et al. Nat Rev Drug Discov 16, 773-786 (2017); doi: 10.1038/nrd.2017.179), methyl binding domains (MBD) (see, Du, et al., Methyl-CpG-binding domain proteins: readers of the epigenome. Epigenomics 2015, 7-1051-73.), and Tandem Tudor and Tudor-like domains (see, Lu et al., Trends Biochem Sci. 2013 November; 38 (11) doi: 10.1016/j.tibs.2013.08.002).

In an example embodiment, the protein domains can be present in the same protein, for example the bromodomain and the PHD domain from BPTF. In an example embodiment, the domains are from the Royal family of chromatin binding domains or are evolutionarily related to the Royal family of binding domains. Example chromatin readers that include domains that can be utilized in the present invention include those described, for example, in Arrowsmith et al., Targeting non-bromodomain chromatin readers. Nat Struct Mol Biol 26, 863-869 (2019); doi: 10.1038/s41594-019-0290-2, incorporated herein by reference in its entirety.

In an embodiment, the recombinant fusion protein, e.g., tandem reader or super reader, comprises one or more chromodomains. Example chromodomains include the chromodomain helicase DNA-binding (CHD) family of proteins, including CHD1, CHD2, CHD3, CHD4, CHD5, CHD6, CHD7, CHD8, CHD9 and chromobox (CBX) 1, CBX2, CBX3, CBX4, CBX5, CBX6, CBX7, CBX8. Other example chromodomains include, for example, M phase phosphoprotein 8 MPP8 (reads H3K9me3), human heterochromatin protein 1 (HP1) (also known as Cbx5) and Drosophila HP1 (dHP1), or of latency associated nuclear antigen (LANA) (see Juillard, et al., Biol. Sci., 117 (36) 22443-22451 (2020); doi: 10.1073/pnas.2004809117). The chromodomain of M phase phosphoprotein 8 MPP8 (reads H3K9me3) is located at residues 55-116, human heterochromatin protein 1 (HP1) (also known as Cbx5) is located at residues 16-77, and Drosophila HP1 (dHP1) is located at residues 20 to 81 of the protein.

In an embodiment, the recombinant fusion protein, e.g., tandem reader or super reader, comprises one or more malignant brain tumor (MBT) domains, which are found in polycomb group (PcG) proteins and bind primarily mono- and di-methylated lysines on histone H3 and H4 tails during posttranslational modification.

In an embodiment, the recombinant fusion protein, e.g., tandem reader or super reader, comprises a methyl binding domains (MBD), for example MeCP2, which is a reader of epigenetic information contained in methylated and hydroxymethylated DNA. Ragione et al., Briefings in Functional Genomics, 15:6, 420-431 (2016); doi: 10.1093/bfgp/elw023.

In an embodiment, the recombinant fusion protein, e.g., tandem reader or super reader, comprises one or more bromodomains, which serve as readers of histone acetylation marks regulating the transcription of target promoters and/or enhancers. Example bromodomains include domains from BRDT bromodomains 1 and 2, TRIM33 bromodomain, BRD4 bromodomains 1 and 2, BRD3 bromodomains 1 and 2, ASH1L bromodomain, SP140 bromodomain, TRIM28 bromodomain, BRD7, TAF1 bromodomains 1 and 2, ATAD2, CECR2 bromodomain, PCAF, BPTF, BAZ2B bromodomain, BRPF3 bromodomain 1, polybromo-1D bromodomain 1, TRIM24 bromodomain, CREB-binding protein bromodomain, BRD9, BRG1, BRD1, WDR9 bromodomain 2, and BRD2 bromodomains 1 and 2. In one embodiment, the bromodomain is from bromodomain PHD finger transcription factor (BPTF), a subunit of NURF chromatin.

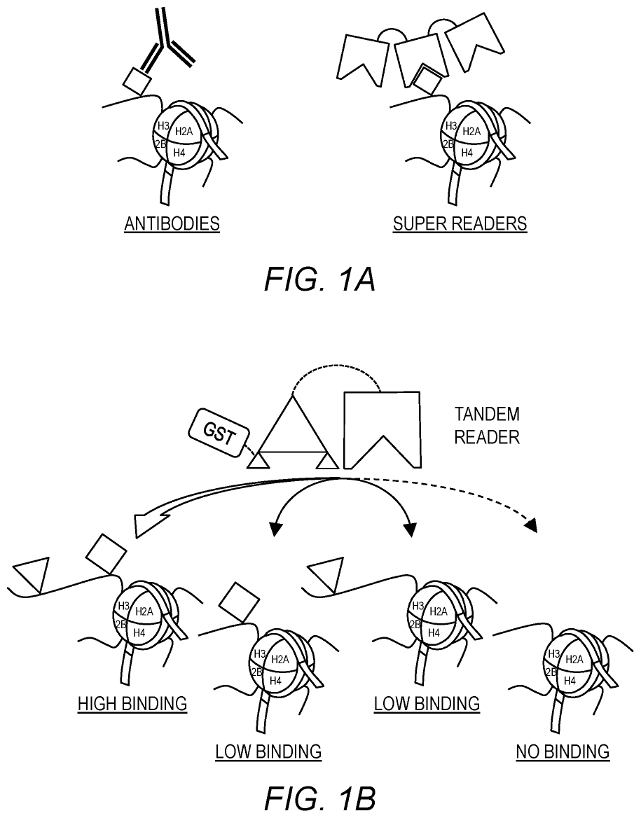

In an embodiment, the recombinant fusion protein, e.g., tandem reader or super reader, comprises one or more PWWP domains, a weakly conserved sequence motif found in >60 eukaryotic proteins that functions as a chromatin methylation reader by recognizing both DNA and histone methylated lysines. Example PWWP domains include DNMT3A, hepatoma-4derived growth factor (HDGF), Pdp2, pdp1, BRDF1, BRF2, BRFF3, HDGF-2, LEDGF/p75, HRP-6, HDGF, MSH6, ZMYND11, MUM1. See, e.g., FIG. 1 of Rona et al., Biophys Rev. 2016 March; 8 (1): 63-74; doi: 10.1007/s12551-015-0190-6 (providing sequence alignment of PWWP domains). In one embodiment, the PWWP domain is DNA methyltransferases DNMT3A.

In an embodiment, the recombinant fusion protein, e.g., tandem reader or super reader, comprises one or more plant homeodomain (PHD) fingers, which are central readers of histone post-translational modifications (PTMs). Example PHD domains include ING1-5, DIDO1, TAF3, RAG2, MORC3, BPTF, KDM4C, KDM5A, KDM7A, PHF2, PHF8, PHF13, PHF23, MLL5, KDM5A, KDM5B, PHF21A, CHD4, CHD5, DPF2, AIRE, TRIM66, SP140L, BAZ2B, PHRF1, CHD5, KDM5B, SP140 PHD, and TRIM28. In one embodiment, the PHD is from bromodomain PHD finger transcription factor (BPTF), a subunit of NURF chromatin. In one embodiment, the PHD domains is from the TAF3 protein.

In embodiments, the improved detection reagents comprise a recombinant fusion protein comprising two or more different protein domains, also referred to as tandem readers. The recombinant fusion proteins can be used in genomic assays, including in genomic mapping assays such as CUT&RUN and CUT&Tag. Tandem readers can be generated using two different domains from the same chromatin reader protein, which may be referred to as a “Natural” Tandem Readers; e.g., domains from the same protein, for example, PHD, which recognizes H3K4me3+bromodomain, recognizing H3 poly-acetyl of BPTF. Tandem readers can also be generated using two domains from two or more different binding domains, also referred to “Artificial” Tandem Readers; e.g., chromodomain of MPP8, which reads H3K9me3+PWWP domain of DNMT3A, which reads H3K36me2/3). In one embodiment, the tandem reader further comprises a label, for example, an epitope tag as described herein. In one embodiment, the tandem reader uses naturally linked chromatin reader domains PHD and bromodomain derived from the protein BPTF with an N-terminal GST epitope tag.

In an embodiment, the tandem readers can map two or more modifications on a single nucleosome or adjacent nucleosomes. In some embodiments, the tandem readers are used in genomics assays to map the location of two or more chromatin features, such as a histone PTMs, transcription factors, and/or DNA modifications.

In some embodiments, tandem readers combine reader domains that recognize a defined single histone or DNA modification of interest (or other nucleosome structures such as the acid patch) to create recombinant dual-reader proteins that cooperatively engage dual PTMs of interest (FIG. 1B). In an embodiments, natural Tandem Readers and artificial Tandem Readers are each capable of specifically binding to dually modified nucleosomes. The optimized fusion proteins identified by the methods provided herein can be capable of specifically binding to two or more chromatin targets with increased binding efficiency and/or specificity of at least 5-fold relative to a single chromatin target. For example, as shown in the working examples, the tandem readers are capable of specifically binding to dually modified nucleosomes at 5-fold, 10-fold, 20-fold, 30-fold, 40-fold, 50-fold, 60-fold, 70-fold, 80-fold, or 90-fold increased binding specificity and/or efficiency or more versus a single chromatin target.

In an embodiment, the tandem reader can be characterized using recombinant designer nucleosome (dNuc) technology, which enables characterization of tandem reader binding against physiological nucleosome substrates carrying single or multiple histone PTMs and/or DNA modifications. In some embodiments, a DNA-barcoded nucleosome panel can be designed to contain single and dual modifications and is used to optimize binding conditions to ensure that a Tandem Reader has a preference (i.e., increase in binding specificity and/or efficiency) for a specific dually modified nucleosome over singly modified nucleosomes.

In an embodiment, the improved detection reagents comprise a recombinant fusion protein comprised of two or more of the same reader protein domains or functional fragments thereof, also referred to as super readers, that can be utilized in genomic mapping assays such as CUT&RUN and CUT&Tag. In an embodiment, the super readers improve assay sensitivity and/or efficiency. The methods provided herein can comprise a recombinant fusion protein with binding of the fusion protein increased by at least 5-fold preference for the chromatin element relative to an off-target chromatin element.

In an embodiment, the super reader comprises 1, 2, 3 or 4 of the same protein domains, In one embodiment, the super reader comprises 2 or more PHD domains. In an embodiment, the super reader comprises one or more H3K4me3 reader TAF3 PHD domains, e.g., 1, 2, or 3 domains. In one embodiment, the super reader comprises a multimer of a chromodomain. In one embodiment, the super reader comprises multimers of the chromodomain helicase DNA-binding (CHD) of CBX1. In one embodiment, the super reader further comprises one or more labels, for example, epitope tags. In an embodiment, the super reader comprises an N-terminal GST tag.

Linkers utilized in the optimized recombinant fusion proteins of the invention can be utilized to link protein domains together and may also be used to link a protein domain and a label. The linkers can be rigid or flexible. The linkers may also comprise a shorter versus a longer sequence for joining together two or more protein domains. Example recombinant fusion protein linkers are described in the art, and are described, for example, in Chen, et al., Adv Drug Deliv Rev. 2013 Oct. 15; 65 (10): 1357-1369, with specific reference to Tables 1 and 3, which is incorporated herein by reference in its entirety. Example flexible linkers include (GGGGS)ₙ (SEQ ID NO:1), wherein n is between 1 and 4 and (Gly) n (SEQ ID NO:2) wherein n is between 4 and 10. Rigid linkers may be designed to adopt an alpha helical conformation. Example rigid linkers can comprise (EAAAK)ₙ (SEQ ID NO:3) wherein n is between 1 and 4, or (XP) n (SEQ ID NO: 4), wherein n is between 1 and 10, where X is any amino acid, preferably wherein each X is independently Ala, Lys or Glu. Another example linker is the linker sequence from DNMT3A, as it naturally occurs, or further modified with a flexible linker, for example, (GGGGS)ₙ (SEQ ID NO: 1), wherein n is between 1 and 4. Additional linkers are provided in the sequences in Table 1. In some embodiments, the recombinant protein may further comprise one or more recognition sequences, for example, protease cleave sites, adjacent to the linker in some instances.

Labels may be included in the recombinant fusion protein. In an embodiment, the label is an epitope tag. Epitope tags may be included in the recombinant fusion protein. Epitope tags can be leveraged, for example, for immunoprecipitation in ChIP-seq, or targeted digestion/tagmentation in assays such as CUT&RUN and CUT&Tag. In one example embodiment, the epitope tag is GST, which can be detected in various genomics workflows (e.g., ChIP, CUT&RUN, CUT&Tag, etc.) using an anti-GST antibody. Affinity tags such as 6×His and maltose binding protein (MBP) as well as peptide tags such as Hemagglutinin (HA), Myc, FLAG, TAP, and V5 can be used in conjunction with corresponding anti-epitope antibodies and will be dependent on the end use.

Methods for screening a detection reagent for a chromatin element for desired characteristics, e.g., specificity and/or efficiency, are provided, comprising: providing a panel of recombinant nucleosomes, each nucleosome comprising one or more chromatin elements recognized by the one or more binding domains present in the detection reagent and/or one or more chromatin elements not recognized by the one or more binding domains present in the detection reagent, thereby providing both on-target and off-target recombinant nucleosomes; and performing a genomic assay with the panel of recombinant nucleosomes to identify the binding specificity and/or efficiency of the detection reagent. In one embodiment, the detection reagent is a recombinant fusion protein comprised of one or more binding domains that recognize one or more of the chromatin elements of at least one of the nucleosomes in the panel, and a label. The genomic assay can comprise a genomic mapping assay such as CUT&RUN or a binding assay, such as a proximity bead-based assay such as dCypher® (see, Marunde, et al., (2022). The dCypher Approach to Interrogate Chromatin Reader Activity Against Posttranslational Modification-Defined Histone Peptides and Nucleosomes. In: Horsfield, J., Marsman, J. (eds) Chromatin. Methods in Molecular Biology, vol 2458. Humana, New York, NY; doi: 10.1007/978-1-0716-2140-0_13, incorporated herein by reference in its entirety) or a bead-based multiplex assay such as Luminex®-dCypher® assay.

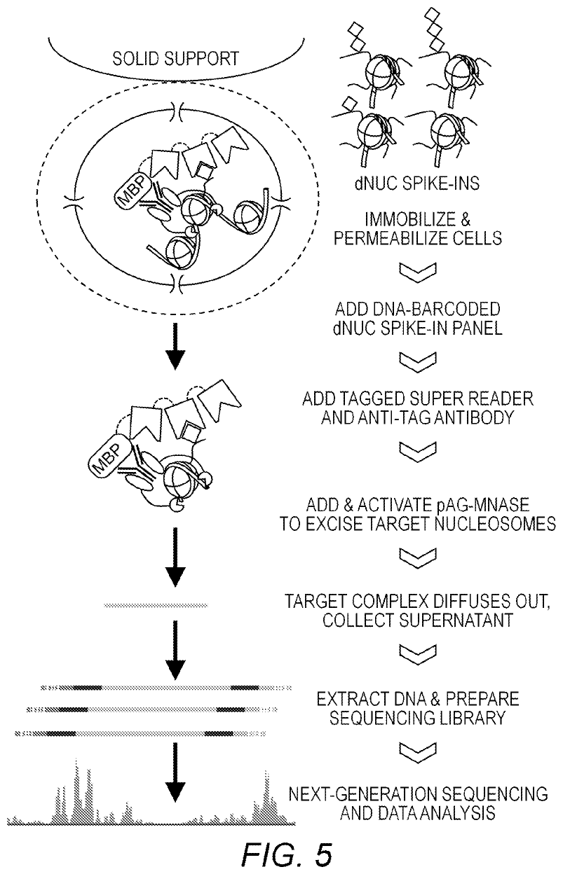

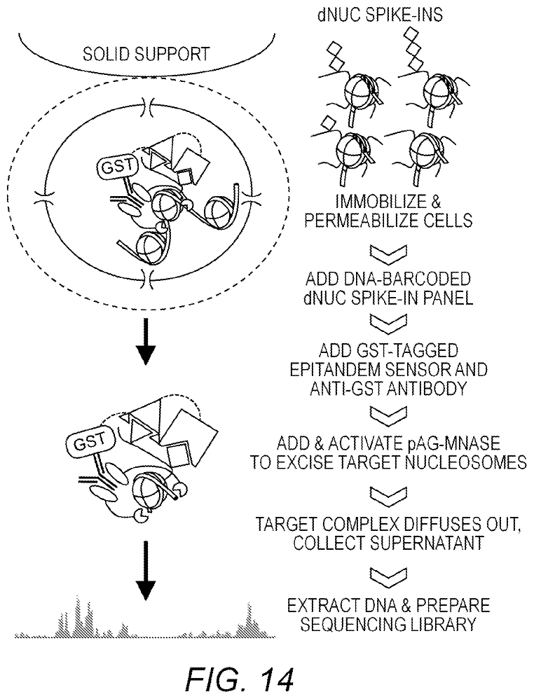

An example method for screening the detection reagents of the present invention include the CUT&RUN workflow depicted in FIG. 5 for a fusion protein comprising two or more of the same binding domains. The steps include capturing cells on a solid support, for example magnetic beads. Adding a nucleosome panel of controls can then be performed to thereby enable optional quality control testing at one or more, or each, subsequent step of the assay. Permeabilizing the cells and incubating the cells with the labeled detection reagent for a chromatin element is performed. A binding pair to the labeled detection reagent is then incubated with the cells, followed by excising of target nucleosomes, for example, by adding and activating an enzyme to excise target nucleosomes, which may comprise linker DNA to allow efficient nuclease cleavage, thereby releasing the DNA-barcoded dNuc from the solid support, with the target dNuc diffusing out and collected in supernatant. Extracting the DNA and preparing a sequencing library for next generation sequencing can then be performed to identify chromatin element binding. Similarly, as shown in FIG. 14, CUT&RUN can be used with recombinant fusion proteins comprising two or more different binding domains, referred to as an EpTandem Sensor in FIG. 14. Immobilizing cells, adding a panel of nucleosomes for quality control, permeabilizing the cells, and incubating with the recombinant fusion protein comprising a label is performed. A binding partner to the protein label and an enzyme capable of binding to the binding partner are then added—in FIG. 14, this is an anti-GST antibody, which is an anti-epitope antibody to the epitope label of the recombinant fusion protein, and pAG-MNase that binds to the anti-GST antibody, respectively. Activating the enzyme thereby fragmenting DNA from cells and dNucs is collected in supernatant and sequenced and analyzed to identify binding of the recombinant fusion protein to chromatin elements. When sued as a screening method for identifying recombinant fusion proteins with desired specificity and/or efficiency, the sequencing step identifies binding preferences and/or validate specificity of the recombinant fusion protein. In embodiments, the genomic mapping assays when used in screening can further comprise background control reactions including, for example, anti-label without recombinant protein, anti-label antibodies, as well as highly specific antibodies to individual chromatin elements, for example, histone PTMs.

In an embodiment, optimization of detection reagents, including the recombinant fusion proteins described herein, comprise use of recombinant modified nucleosomes, carrying one or more chromatin elements, e.g., a histone and/or DNA modifications, histone mutations, histone variants, or proteins that directly or indirectly bind chromatin, to systematically optimize the use of the recombinant fusion proteins as detection reagents for chromatin assays.

In some embodiments, recombinant nucleosome substrates are utilized to optimize engineering of the recombinant fusion protein, including binding domain(s) selection, epitope tag (e.g., GST, His, FLAG®), epitope tag position (e.g., C-terminus vs. N-terminus), linker sequence (e.g., flexible, rigid, short, long), and other reaction components and conditions including buffer conditions (e.g., salt concentration). As shown in the working examples, each of these parameters can have major impacts on the binding of recombinant protein domains on nucleosomes. Differences in binding properties between histone peptides and recombinant nucleosomes have been previously shown for antibodies, which are large multidomain proteins. Importantly, these nucleosome binding properties are not recapitulated on histone peptides (the current gold standard for detection reagent characterization) (Shah, et al., Mol Cell. 2018 Oct. 4; 72 (1): 162-177.e7; doi: 10.1016/j.molcel.2018.08.015), demonstrating the requirement to use fully defined recombinant nucleosome substrates for binding domain characterization/optimization.

### Nucleosomes

Nucleosomes useful in the present invention include recombinant nucleosome substrates and DNA-barcoded nucleosomes as spike-in controls for epigenomic mapping assays. Example DNA-barcoded nucleosomes as spike-in controls for epigenomic assays include those described in International Patent Publication Nos. WO 2020/132388, WO 2020/168151, WO 2019/140082, WO 2013/184930, WO 2015/117145, each of which is incorporated herein in their entirety.

Reagents comprised of pools or panels of DNA-barcoded designer nucleosomes (dNucs) carrying on- and related off-target epitopes (e.g., histone PTMs or chromatin binding domain epitopes) may be used as controls to directly assess detection reagent (e.g., antibody) performance as well as the performance of the recombinant fusion proteins for improved and or optimized assays. The barcode sequence and methods of incorporation and use can be as described in International Patent Publication No. WO 2019/140082 and International Patent Publication WO 2020/132388, incorporated herein by reference. Barcoding can be used as needed to identify the cleaved nucleosomal DNA, for example by sample, individual, or other source identifying information.

A recombinant nucleosome can comprise one or more chromatin elements. Chromatin elements as used herein include histone modifications, histone mutations or histone variants, DNA modifications, or proteins that directly or indirectly bind chromatin. In some embodiments, the chromatin elements comprise proteins that bind unmodified histones. In an aspect, the unmodified histone is the acid binding patch, a set of negatively charged amino acids clustered on the surface of the nucleosome disk at the interface of histones H2A and H2B. The chromatin elements can be comprised of both histone modifications and proteins that directly or indirectly bind chromatin.

A recombinant nucleosome substrate may comprise a recombinant mononucleosome. The recombinant nucleosome substrate may comprise one or more histone modification and/or DNA modification, i.e., is functionalized. A recombinant nucleosome may comprise a protein octamer, containing two copies each of histones H2A, H2B, H3, and H4, and optionally, linker histone HI. Each of the histones in the nucleosome is independently fully synthetic, semisynthetic, or recombinant. Methods of producing histones synthetically, semi-synthetically, or recombinantly are well known in the art.

In an aspect, the histone can comprise one or more post-translational modification. The histone PTM may be any PTM for which measurement is desirable. In some embodiments, the histone PTM is, without limitation, N-acetylation of serine and alanine; phosphorylation of serine, threonine and tyrosine; N-crotonylation, N-acylation of lysine; N6-methylation, N6,N6-dimethylation, N6,N6,N6-trimethylation of lysine; omega-N-methylation, symmetrical-dimethylation, asymmetrical-dimethylation of arginine; citrullination of arginine; ubiquitinylation of lysine; sumoylation of lysine; O-methylation of serine and threonine, ADP-ribosylation of arginine, aspartic acid and glutamic acid, or any combination thereof.

In one embodiment, the post translational modification is selected from one or a combination of modifications listed in Tables 1(a)-1(f) of International Patent Publication WO 2019/169263, specifically incorporated herein by reference.

The histone mutation may be any mutation known in the art or any mutation of interest. In some embodiments, the histone mutations are oncogenic mutations, e.g., mutations associated with one or more types of cancer. Known oncogenic histone mutations include, without limitation, H3K4M, H3K9M, H3K27M, H3G34R, H3G34V, H3G34W, H3K36M, or any combination thereof.

Several naturally occurring histone variants are known in the art and any one or more of them may be included in the nucleosome. Histone variants include, without limitation, H3.3, H2A.Bbd, H2A.Z.1, H2A.Z.2, H2A.X, mH2A1.1, mH2A1.2, mH2A2, TH2B, or any combination thereof.

The DNA post-transcriptional modification may be any modification for which measurement is desirable. Known post-transcriptional DNA modifications include, without limitation, 5-methylcytosine, 5-hydroxymethylcytosine, 5-formylcytosine, 5-carboxylcytosine, 3-methylcytosine, 5,6-dihydrouracil, 7-methylguanosine, xanthosine, and inosine. In some embodiments, the DNA post-transcriptional modification is 5-methylcytosine, 5-hydroxymethylcytosine, 5-formylcytosine, 5-carboxylcytosine, 3-methylcytosine, or any combination thereof.

The recombinant mononucleosome may comprise a mix of recombinant and/or synthetic histone octamers, one or more of which may comprise post-translational modifications (PTMs). In some embodiments, the recombinant nucleosomes are polynucleosomes comprising a plurality of octamers. In an aspect, the polynucleosome comprises less than 6, 5, 4, or 3 histone octamers. In some embodiments, each histone octamer comprises the same PTM(s), e.g., the nucleosomes are homogenous. In other embodiments each histone octamer comprises different PTM(s), e.g., the nucleosomes are heterogeneous. The recombinant mononucleosomes can be as described in International Patent Publication No. WO 2018/213719, incorporated herein by reference in its entirety.

One preferred aspect of the invention is the use of recombinant substrates, which can be manufactured to contain one (or more) physiological or disease-relevant histone and/or DNA modifications.

In some embodiments, the method for screening a detection reagent (e.g., a recombinant fusion protein detection reagent) in an optimization assay comprises use of a panel (e.g., a plurality) of spike-in nucleosomes. The number of different nucleosomes in a panel can include a plurality of species, which may include duplicates of each standard having distinct barcode identifier sequences as a form of internal control. Thus, the panel may include species represented multiple times at the same or different concentrations with each standard having a unique barcode identifier sequence that represents the concentration of the standard. For example, each standard may be present in the panel in 2, 3, 4, 5, 6, 7, 8, 9, 10 or more different concentrations, each concentration having a different barcode identifier sequence. Thus, a panel may have unique standards of between about 5 and about 250 total species. Example methods of use are described in the working examples, as well as International Patent Publication WO 2019/140082, incorporated herein by reference in its entirety.

A further aspect of the invention relates to kits for use in the methods of the invention. Kits may comprise one or more recombinant nucleosome panels; optionally nucleosome spike-ins, and one or more recombinant fusion proteins; one or more solid supports; and/or instructions for use, in any combination. The kit can further comprise modulating agents, carriers, buffers, containers, devices for administration of the components, and the like. The kit can further comprise labels and/or instructions for assay selection and execution. Such labeling and/or instructions can include, for example, information concerning the amount, and method of administration, detection and quantification for the assays detailed herein.

Having described the present invention, the same will be explained in greater detail in the following examples, which are included herein for illustration purposes only, and which are not intended to be limiting to the invention.

### EXAMPLES

### Example 1. Development and Validation of H3K9Me3 Super Reader

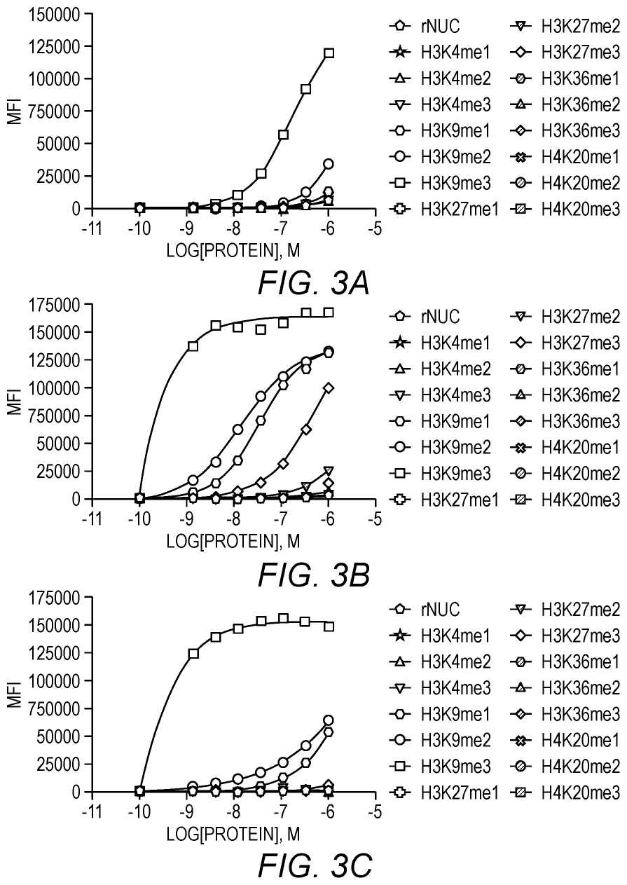

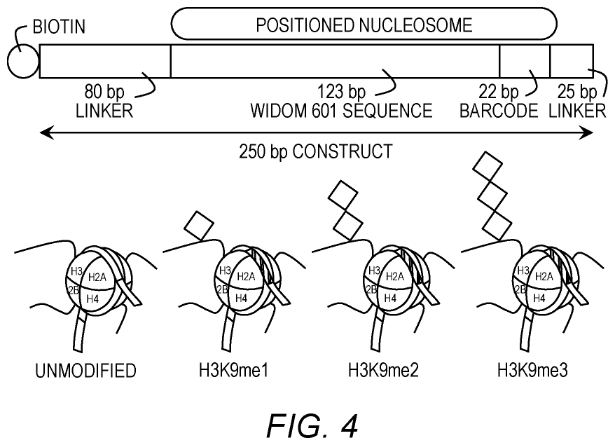

The H3K9me3 Super Reader was developed by multimerizing the chromodomain helicase DNA-binding (CHD) of CBX1. To interrogate the effects of multimerization on binding affinity and specificity across a diverse set of dNuc substrates, constructs with 1×, 2×, and 3× linked reader domains (CBX1 CHD) were generated in series and their binding activity profiled with dCypher assays (FIGS. 3A-3B). Reader constructs were generated with an N-terminal MBP tag and confirmed purity (>80% by analytical HPLC and Coomassie SDS-PAGE). A flexible 2×(GGGGS) (SEQ ID NO:5) linker was used for the 2× and 3× Readers. It was observed that, compared to the single reader domain, 3× multimerization of the CBX1 CHD improved on-target binding activity >100-fold with minimal change in off-target binding (FIGS. 3A, 3C). Interestingly, while the 2× construct also showed a >100-fold increase in H3K9me3 binding, it also had higher cross-reactivity (FIGS. 3A,3B). Thus, the 3×CBX1 CHD construct was selected as the H3K9me3 Super Reader for further characterization.

### Example 2: Development of Adapted CUT&RUN Workflow Using H3K9Me3 Super Reader

A modified version of a high-performance CUT&RUN workflow was developed to map H3K9me3 with a Super Reader, demonstrating the utility of Super Readers as detection reagents for genomic mapping. Applicant demonstrated that the H3K9me3 Super Reader is highly specific for its PTM target in a CUT&RUN workflow and generates high-quality, reliable genomic profiling data that is indistinguishable from data obtained from highly validated and specific antibodies.

Development of CUT&RUN-compatible dNuc spike-in controls: A CUT&RUN-compatible spike-in panel of DNA-barcoded dNucs carrying all methylation states at H3K9 (me0, me1, me2, and me3) was developed (Error! Reference source not found.4), which was used to confirm that the H3K9me3 Super Reader was specifically targeting the trimethyl mark in the modified CUT&RUN assay. To generate these CUT&RUN-compatible dNucs, a proprietary nucleosome manufacturing pipeline was used to develop modified histones, which were assembled into octamers and wrapped with barcoded DNAs that included a biotin tag for immobilization on streptavidin-coated magnetic beads. Templates contained additional linker DNA to allow efficient micrococcal nuclease (MNase) cleavage, which releases the DNA-barcoded dNuc from the solid support.

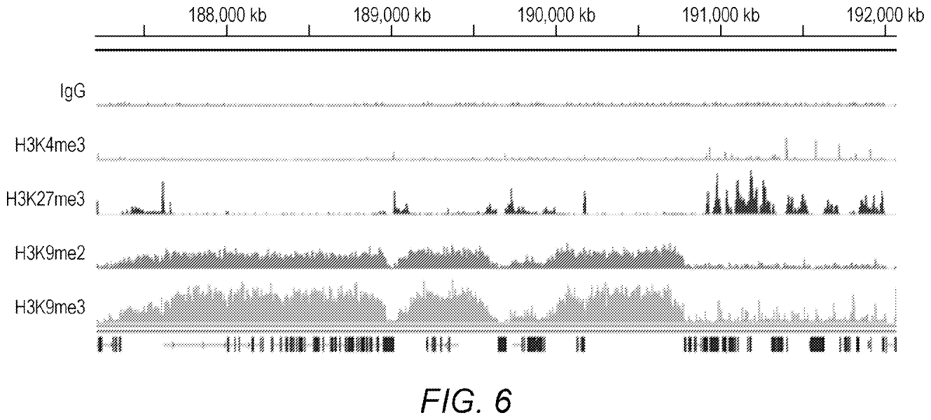

Development of adapted CUT&RUN workflow with Super Reader and dNuc spike-in controls: Applicant adapted the standard CUT&RUN approach to validate the performance of the H3K9me3 Super Reader for genomic mapping. Briefly, 500,000 K562 cells were harvested and immobilized on concanavalin A (ConA) magnetic beads. Of note, the spike-in dNuc panel was added at this step, thus enabling quality control testing at each step of the assay. Cells were permeabilized, and then incubated with the MBP-tagged H3K9me3 Super Reader, followed by an anti-MBP antibody and pAG-MNase (which binds to the anti-MBP antibody) (see FIG. 5 for assay workflow). Finally, samples were treated with pAG-MNase, and antibody-directed MNase cleavage activated by the addition of calcium. Fragmented DNA (from cells and spike-in dNucs) was collected from the supernatant and prepared for sequencing using the NEBNext Ultra II Library Prep Kit, and sequenced on the Illumina MiniSeq, generating ˜6 million paired end reads per sample. Data were analyzed using applicant's internal analysis pipeline based on published work. To rigorously assess performance of the Super Reader, a series of background control CUT&RUN assays were also conducted using IgG as well as recombinant MBP+anti-MBP (no Reader). For benchmarking, CUT&RUN was also performed with the best-in-class H3K9me3 antibody, which has been shown to generate high quality data in CUT&RUN (FIG. 6).

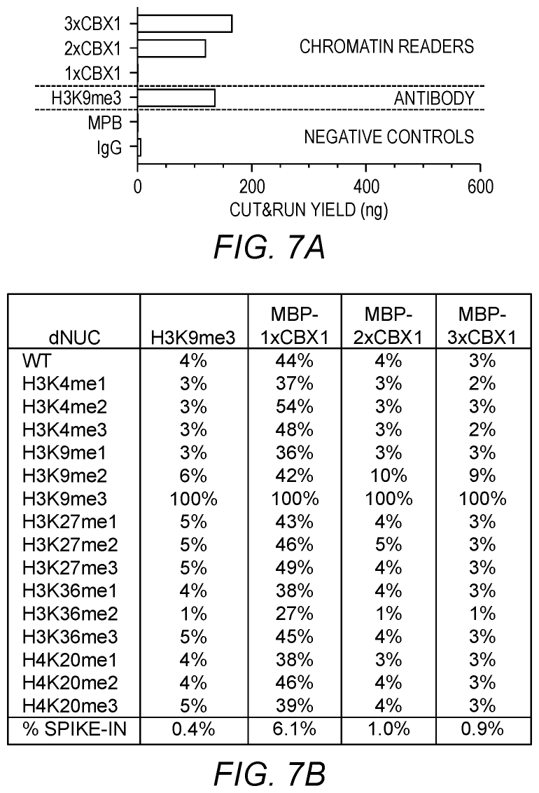

The total yield of DNA recovered by the H3K9me3 Super Reader (prior to library preparation) was first compared and it was found that it recovered more DNA relative to the H3K9me3 antibody (FIG. 7A). Next, recovery of the DNA-barcoded spike-in dNucs was analyzed to validate the specificity of the Super Reader in CUT&RUN. The H3K9me3 Super Reader displayed >10-fold preference for its target, indicating very high specificity (well over the >5-fold specificity threshold used for PTM mapping with antibodies) (FIG. 7B). Notably, multimerization of the CBX1 CHD dramatically improved DNA yield and specificity compared to a single domain, highlighting the power of the present approach. The control reactions behaved as expected, with low DNA yields and nonspecific recovery of all spike-ins. Taken together, these data demonstrate that EpiCypher's dNuc spike-in technology can be used to determine Super Reader binding performance and specificity in CUT&RUN, and confirm that the H3K9me3 Super Reader specifically recovers on-target nucleosomes in this assay.

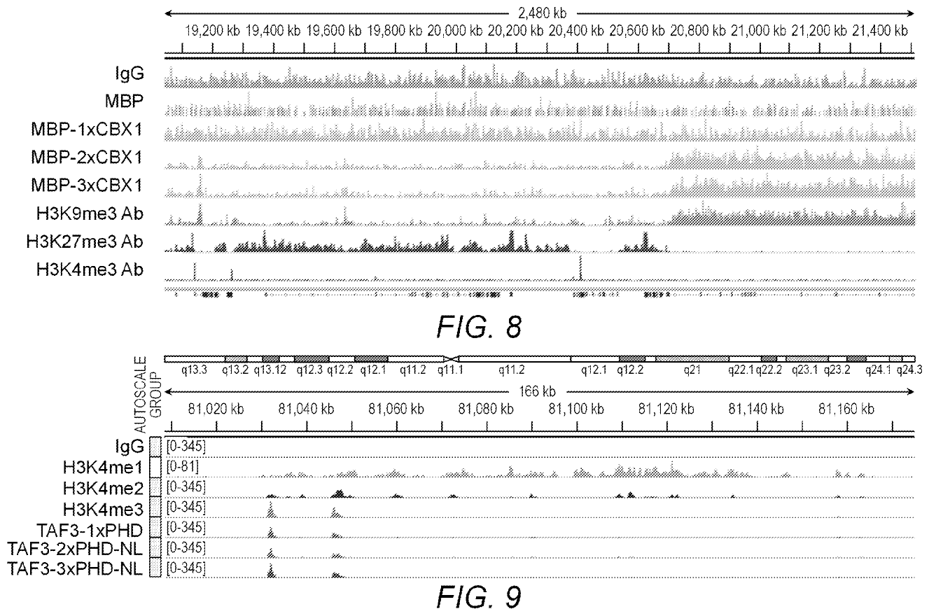

Application of Super Reader for genomic mapping: Next, the data generated with the adapted CUT&RUN workflow above was utilized to map H3K9me3. Briefly, raw reads were mapped to the reference human genome (hg19) using Bowtie2. Resulting SAM files were filtered using SAMtools, and BEDTools, which were used to create genome coverage BEDgraphs. SEACR (Sparse Enrichment Analysis of CUT&RUN) was used to call peaks. Genome-wide peaks were compared between the H3K9me3 Super Reader and the validated H3K9me3 antibody and it was observed that the Super Reader faithfully recapitulated the same peaks as the antibody, further validating its performance and specificity (FIG. 8). The IgG and MBP negative controls showed only background signal, and the single reader domain also did not show any significant signal or significant enrichment in the expected areas. These data demonstrate that multimerized chromatin reader domains (i.e., Super Readers) may be utilized as high-performance detection reagents for PTM mapping in genomic workflows and provide strong proof-of-principle for the present invention.

Applicant successfully identified highly specific chromatin reader domains for challenging PTM targets, using the high throughput dCypher assay to characterize the binding activity of these reagents on dNuc substrates. A H3K9me3 Super Reader was developed and its high affinity and specificity was demonstrated. Further, the H3K9me3 Super Reader was applied for PTM profiling in a modified CUT&RUN workflow, demonstrating the potential of these novel detection reagents to provide a pathway to study challenging and new PTM targets.

### Example 3. Demonstration that Tags Impact Super Reader Binding in CUT&RUN

The 1×CBX1 construct which contained an N-terminal MBP tag did not function well in CUT&RUN workflows (FIG. 8). This example shows the impact of protein tags on domain function. Notably, it was determined that it is advantageous to use tags that have the ability to dimerize (e.g., Glutathione S-Transferase [GST] tag vs. Maltose Binding Protein [MBP] tag). The present data suggests that one can artificially circumvent having to multimerize domains by including a tag that will force domains into close proximity and act through pseudo-multimerization. In this example the H3K4me3 reader TAF3 PHD was used, which was designed to contain an N-terminal GST tag. The construct was also multimerized (e.g., 1×, 2×, and 3×) and then submitted for analysis in a CUT&RUN workflow with spike-in controls that contained H3K4me3 modifications as described above. In this case, the GST-tagged 1×TAF3 still was able to bind specifically to its cognate target in vivo as compared to the best-in-class, highly validated H3Kme3 antibody and showed preferred specificity toward the H3K4me3 barcoded nucleosomes. These data together suggest not only may domain multimerization lead to a functional reagent, but the protein tag can also influence specificity and function in CUT&RUN workflows.

### Example 4. Development of the BPTF Tandem Reader Sensor

Applicant created two recombinant Tandem Reader Sensors as novel combinatorial PTM detection reagents and characterized their binding activity on dNuc substrates in biochemical assays. The importance of the nucleosome context for binding (vs. histone peptides) was demonstrated and validated the specificity of the Tandem Reader Sensors for combinatorial PTMs (vs. single PTMs) on dNuc substrates.

The first Tandem Reader Sensor was developed using two naturally linked chromatin reader domains derived from the protein BPTF: PHD (reads H3K4me3) and Bromo (reads H3 acetylation). BPTF is a subunit of the NURF chromatin remodeling complex and has been associated with neurological and developmental disorders as well as numerous cancers. The recombinant BPTF Sensor was generated with an N-terminal Glutathione S-Transferase (GST) tag (essential for compatibility with downstream biochemical and CUT&RUN assays) and was confirmed for identity (by immunoblot with BPTF antibody) and purity (>80% by analytical HPLC and Coomassie SDS-PAGE).

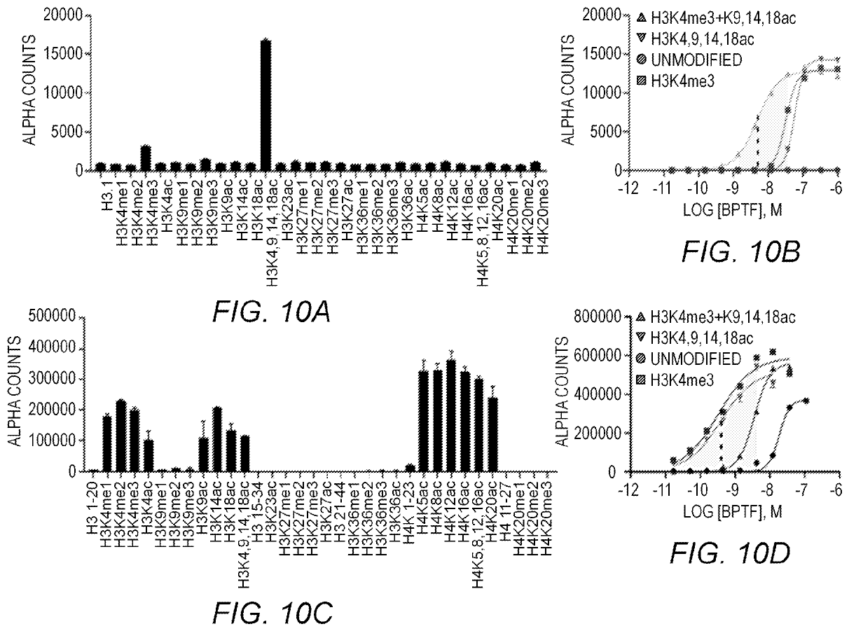

To assess the binding specificity of the BPTF Tandem Reader Sensor, its binding was first characterized across single PTMs. The high-throughput dCypher platform was utilized to screen BPTF Tandem Reader sensor binding across diverse panels of dNucs and histone peptides (FIGS. 10A-10D). For this bead-based proximity assay, the GST-tagged BPTF Sensor was coupled to glutathione acceptor beads and probed against biotinylated dNucs or histone peptides coupled to streptavidin donor beads. In preliminary studies, the BPTF Tandem Reader sensor was first screened against 65 dNucs and 288 modified peptides; a subset of results are shown in FIG. 10A and FIG. 10C, respectively. On dNuc substrates, binding of the BPTF Tandem Reader sensor against H3K4me3 and poly-acetylated H3 (H3K4,9,14,18ac) was observed. Interestingly, the Tandem Reader did not bind to individual H3 acetylated marks, suggesting that the BPTF bromodomain itself engages in multivalent interactions across combinatorial H3 acetylated marks. In contrast to published data using histone peptides the BPTF Tandem Reader binding against H4 acetylated dNucs was not observed. On histone peptides, the BPTF Tandem Reader showed nonspecific binding across multiple H3 methyl and acetyl marks, as well as H4 acetylation. These data highlight the importance of screening across a diverse range of dNuc substrates (vs. histone peptides) to thoroughly interrogate Tandem Reader and by extension Super Reader binding in the physiologically-relevant nucleosome context.

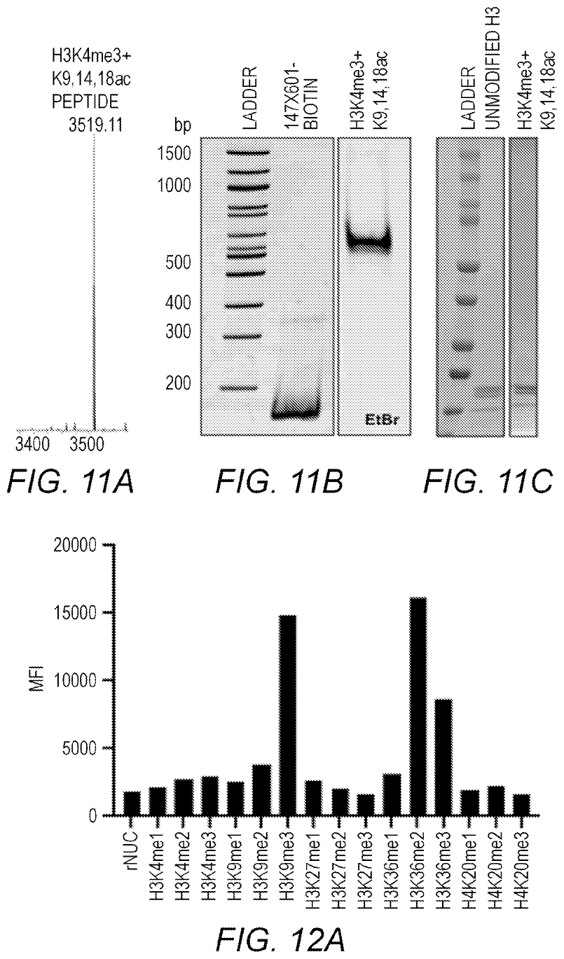

Interrogation of BPTF Tandem Reader binding specificity using a combinatorial dNuc: To demonstrate the specificity of the BPTF Tandem Reader for dual vs. single PTMs, a novel combinatorial dNuc was generated carrying the specific dual PTMs indicated by the preliminary screen (H3K4me3 and H3 poly-acetylation). Combinatorial dNucs were assembled using Applicant's proprietary dNuc manufacturing pipeline. Briefly, a “traceless” native chemical ligation-based strategy was used to develop modified histones, which were assembled into octamers/nucleosomes with DNA templates containing the 147 bp Widom 601 sequence and a biotin tag. Modified histone peptides were verified to be >95% pure using HPLC and identity confirmed by mass spectrometry (FIG. 11A). Native PAGE was used to confirm absence of free DNA (FIG. 11B) and SDS-PAGE Coomassie staining to validate histone stoichiometry (FIG. 11C) in the final dNuc preparation. PTM identity was confirmed by immunoblot.

To determine binding sensitivity, titrations of the BPTF Tandem Reader were performed against the combinatorially-modified dNuc and histone peptide, along with the singly-modified substrates (FIG. 10A and FIG. 10D). The BPTF Sensor had similar binding to the H3K4me3 and H3 poly-acetylated dNucs, with apparent EC50s of 77.8 nM and 61.0 nM, respectively. Strikingly, when titrated against the combinatorial H3K4me3+H3 poly-acetylated dNuc, the BPTF sensor exhibited an apparent EC50 of 2.7 nM, demonstrating a synergistic ˜25-fold improvement in binding on the combinatorial dNuc vs. the singly-modified dNucs. Again, it was observed that the dNuc context was critical to observe this binding specificity, as the BPTF Tandem Reader displayed roughly equal binding to the single H3K4me3 and the dual H3K4me3+H3 poly-acetylated histone peptides, as well as nonspecific binding to the unmodified histone peptide (FIG. 10D). The results confirm the essential role of multivalent nucleosome interactions for proper engagement of reader domains and in defining the histone code, and strongly support the development of additional Tandem Reader Sensors (i.e., Tandem Reader Sensor).

### Example 5. Development of an Artificial Tandem Reader Sensor and Combinatorial dNuc and Linker Optimization

As part of this project, Applicant developed artificial Tandem Reader Sensors utilizing reader domains from two different proteins. This type of Sensor design would greatly expand the pool of potential combinatorial PTM targets, as many biologically-relevant dual PTMs are not recognized by known tandem reader proteins. An artificial Tandem Reader Sensor was engineered to target the H3K9me3+H3K36me2/3 combinatorial mark, which marks a novel bivalent chromatin state that is enriched in weakly transcribed chromatin and at ZNF274 and SETDB1 binding sites. Furthermore, these marks are both targeted by the demethylase KDM4A/JMJD2A, a known high-value cancer drug target. To generate the H3K9me3+H3K36me2/3 Tandem Reader Sensor, the chromodomain of MPP8 (reads H3K9me3) and the PWWP domain of DNMT3A (reads H3K36me2/3) were combined. An N-terminal GST tag was included and quality control assessments performed as described above for the BPTF Tandem Reader Sensor. To determine optimal linker length/sequence for multivalent engagement of the Tandem Sensor on nucleosomes, the artificial Sensor was generated with three different linker designs: 1) no linker, 2) the natural DNMT3A linker sequence, and 3) the natural DNMT3A linker plus a flexible glycine and serine linker sequence (GGGGS (SEQ ID NO: 6)). Linker optimization is desirable for Tandem Reader Sensor development, as linkers influence both binding activity and recombinant protein stability. Combinatorial H3K9me3+H3K36me2 and H3K9me3+H3K36me3 dNucs were also assembled, using the same approach and quality control metrics described above.

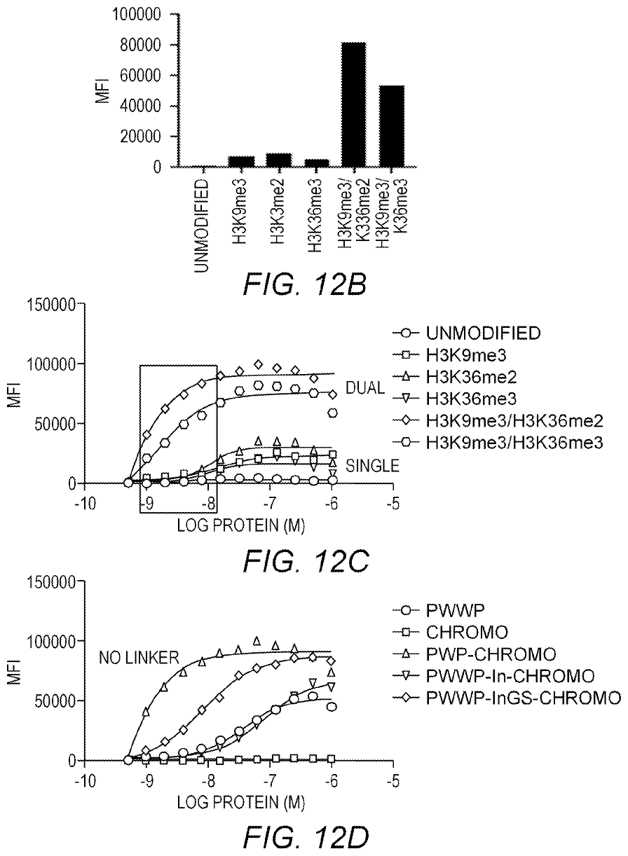

To determine the binding activity of the artificial Tandem Reader Sensor against singly- and combinatorially-modified dNucs, the dCypher approach that was previously used with the BPTF Tandem Reader Sensor was updated. While the first-generation dCypher approach is highly sensitive, it is a “no-wash” assay and may detect relatively weak interactions that do not reflect binding sensitivity/selectivity in downstream assays (e.g., CUT&RUN). To enable sample washing that mimics downstream applications, the recently developed Luminex-based dCypher™ assay was utilized. In this assay, detection reagents (e.g., antibodies or Tandem Reader Sensors) are profiled against a panel of dNucs coupled to spectrally barcoded Luminex xMAP beads, thereby rapidly evaluating binding activity to multiple targets in a single reaction. Notably, dCypher screening is amenable to optimization with various washes to predict binding activity in specific downstream assays (e.g., CUT&RUN/CUT&Tag, ChIP-seq). Here, dCypher was utilized to determine the binding specificity of the artificial Sensor (with three different linker designs) against singly- and combinatorially-modified dNucs (FIGS. 12A-12D). The artificial Sensor recognized H3K9me3 and H3K36me2/3 individually (FIG. 12A) but demonstrated strong preference for the combinatorially-modified dNucs (between 6 to 90-fold change vs. singles; FIGS. 12B, 12C). Surprisingly, the Tandem Reader Sensor without an added linker sequence displayed the strongest binding to the dually-modified dNuc (FIG. 12D). The results show strong proof-of-concept for the present approach in combining independent chromatin reader domains, which can be leveraged to target additional dual PTMs. The work also reveals that the linker identified for joining domains in some instances may not be determined a priori. Linker identity, length, flexibility, and source may have profound effect on Tandem Reader function. Specificity and binding affinity for unique Tandem Readers can be systematically characterized via assays such as our dCypher platform.

### Example 6. Development of Tandem Readers as Detection Reagents in CUT&RUN Using dNuc Spike-In Controls

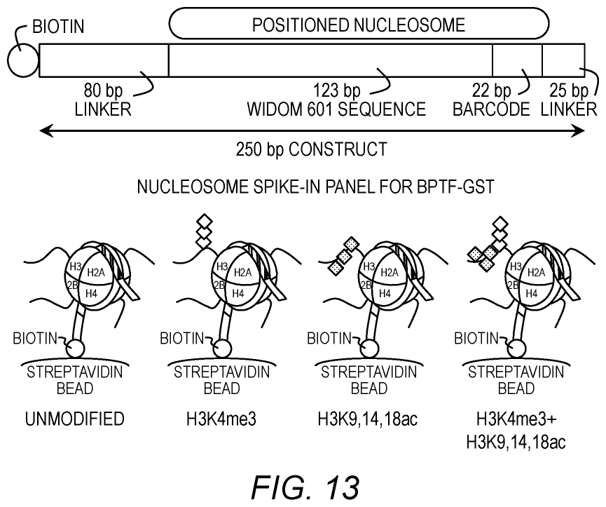

Applicant developed a CUT&RUN-compatible spike-in panel comprising singly- and combinatorially-modified DNA-barcoded dNucs, which was used to confirm that the BPTF Tandem Reader Sensor was specifically targeting combinatorial nucleosomes in vivo (vs. singly-modified; FIG. 13). To generate the panel of dNuc spike-ins for this project, histone octamers were wrapped with barcoded DNAs, including a biotin tag for immobilization on streptavidin-coated magnetic beads. Templates also contained additional linker DNA to allow MNase cleavage, which releases the DNA-barcoded dNuc from the solid support.

Validation of BPTF Tandem Reader Sensor in CUT&RUN with dNuc spike-in controls: The performance of the BPTF Sensor were validated in a genomic mapping application by developing a modified CUT&RUN workflow (FIG. 14). Briefly, 500,000 K562 cells were harvested and immobilized on concanavalin A (ConA) magnetic beads. Of note, the spike-in dNuc panel was added at this step, thus enabling quality control testing at each step of the assay. Cells were permeabilized, and then incubated with BPTF Sensor, followed by an anti-GST antibody and pAG-MNase (which binds to the anti-GST antibody) (see FIG. 14 for example assay workflow). To rigorously assess performance of the Tandem Reader Sensor, a series of background control reactions were also conducted using IgG, anti-GST (no BPTF treatment), and anti-BPTF antibodies, as well as highly specific antibodies to individual histone PTMs (H3K4me3, H3K9ac, H3K18ac). Finally, antibody-directed MNase cleavage was activated by the addition of calcium. Fragmented DNA (from cells and dNucs) was collected from the supernatant and prepared for sequencing using the NEBNext Ultra II Library Prep Kit. Libraries were sequenced on the Illumina MiniSeq, generating ˜6 million paired end reads per sample. Data were analyzed using the internal analysis pipeline based on published work

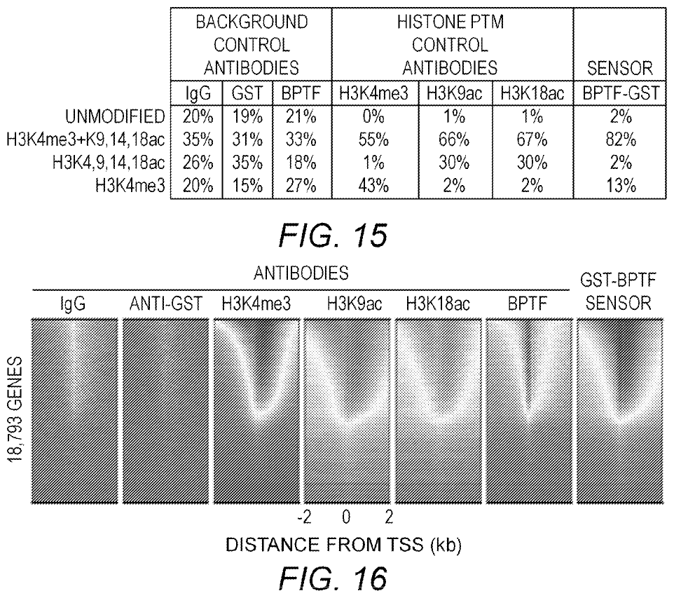

Before examining the distribution of H3K4me3+H3 poly-acetylation genome-wide, recovery of the DNA-barcoded dNucs was first analyzed to validate the specificity of the Tandem Reader Sensor in the CUT&RUN workflow. The BPTF Sensor displayed an 8-fold preference for the combinatorial PTM over either single PTM individually (FIG. 15), indicating its reliability for profiling dual modifications in CUT&RUN assays (well over the >5-fold specificity threshold used for PTM mapping with antibodies). The background control antibodies behaved as expected, with low DNA yields (10-fold less vs. BPTF Sensor) and nonspecific recovery of all four spike-ins. Reactions probed using PTM-specific antibodies enriched for dNucs that contained the target modifications (i.e., H3K4me3 antibody recovered both the H3K4me3 and the combinatorial H3K4me3+H3 poly-acetylated dNucs). Taken together, these data validate the spike-ins panel for application in CUT&RUN assays and confirm that the BPTF Tandem Reader Sensor specifically and preferentially recovers combinatorially-modified nucleosomes.

Application of BPTF Tandem Reader Sensor for genomic mapping: The CUT&RUN data generated above was used to map H3K4me3+H3 poly-acetylation genome-wide. Briefly, raw reads were mapped to the reference human genome (hg19) using Bowtie2 (sensitive preset option, end-to-end alignment). Resulting SAM files were filtered using SAMtools, and BEDTools, which were used to create genome coverage BEDgraphs. SEACR (Sparse Enrichment Analysis of CUT&RUN) was used to call peaks. The anti-IgG and anti-GST negative controls displayed low signal (FIG. 16), indicating low background and highlighting a major advantage of CUT&RUN.

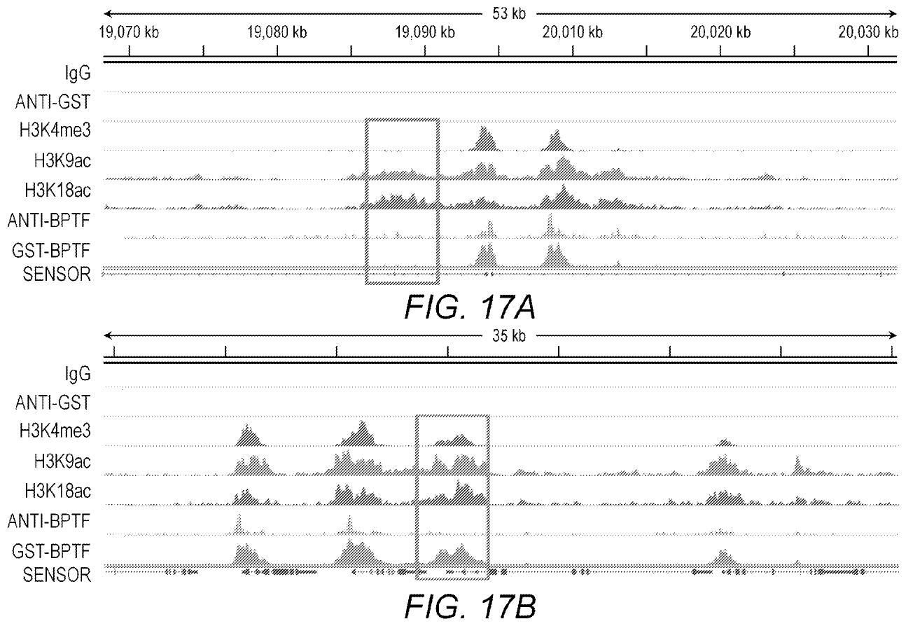

First, the overall signal distribution was compared between the BPTF Tandem Reader Sensor, histone PTMs, and endogenous BPTF, focusing the analysis on promoter regions, where H3K4me3 and many histone acetylation marks are located and BPTF is known to bind (FIG. 9). It was observed that the BPTF Tandem Reader Sensor recapitulated the localization of endogenous BPTF, with a broader distribution that closely matched H3K4me3 and H3 acetyl marks. Next, genome-wide peaks were compared between the BPTF Sensor, histone PTMs, and endogenous BPTF (FIGS. 17A-17B). The BPTF Sensor reliably mapped regions marked by both H3K4me3 and histone acetylation (H3K9ac and H3K18ac included as controls). It was further demonstrated that the BPTF Sensor is highly specific for the combinatorial state, as regions that contain H3 acetylation without accompanying H3K4me3 were not bound by the Tandem Reader Sensor (FIG. 17A; boxed). Importantly, the Tandem Reader BPTF Sensor also bound to H3K4me3+H3 acetylated regions not bound by BPTF (FIG. 17B; boxed). These data demonstrate that the Tandem Sensor detects areas demarcated by these combinatorial PTMs and is not simply replicating full-length BPTF binding (which may be subject to additional regulation as part of the NURF chromatin remodeling complex).

Provided in Table 1 are exemplary recombinant fusion proteins, including proteins utilized in the working examples. Bold and underlined font in the sequences denote binding domains. Underlined sequences denote linkers, which optionally comprise recognition sequences.

TABLE 1
Exemplary Recombinant Fusion Proteins

| Description | SEQUENCE |
|---|---|
| GST- | MSPILGYWKIKGLVQPTRLLLEYLEEKYEEHLYERDEGDKWRNKKFELGLEFPNLPY |
| BPTF(PHD)- | YIDGDVKLTQSMAIIRYIADKHNMLGGCPKERAEISMLEGAVLDIRYGVSRIAYSKDF |
| 1xGS-MPP8 | ETLKVDFLSKLPEMLKMFEDRLCHKTYLNGDHVTHPDFMLYDALDVVLYMDPMCL |
| (CD) | DAFPKLVCFKKRIEAIPQIDKYLKSSKYIAWPLQGWQATFGGGDHPPKSDLEVLFQG |
| | PLGSDTKLYCICKTPYDESKFYIGCDRCQNWYHGRCVGILQSEAELIDEYVCPQ |
| | CQSTEDAMTVLTPLTEKDYEGLKRVLRSLGGGGSDVFEVEKILDMKTEGGKVL |
| | YKVRWKGYTSDDDTWEPEIHLEDCKEVLLEFRKKIAEPGSTRAAAS (SEQ ID |
| | NO: 7) |
| GST- | MSPILGYWKIKGLVQPTRLLLEYLEEKYEEHLYERDEGDKWRNKKFELGLEFPNLPY |
| BPTF(PHD)- | YIDGDVKLTQSMAIIRYIADKHNMLGGCPKERAEISMLEGAVLDIRYGVSRIAYSKDF |
| MPP8 (CD) | ETLKVDFLSKLPEMLKMFEDRLCHKTYLNGDHVTHPDFMLYDALDVVLYMDPMCL |
| | DAFPKLVCFKKRIEAIPQIDKYLKSSKYIAWPLQGWQATFGGGDHPPKSDLEVLFQG |
| | PLGSDTKLYCICKTPYDESKFYIGCDRCQNWYHGRCVGILQSEAELIDEYVCPQ |
| | CQSTEDAMTVLTPLTEKDYEGLKRVLRSLDVFEVEKILDMKTEGGKVLYKVR |
| | WKGYTSDDDTWEPEIHLEDCKEVLLEFRKKIAEPGSTRAAAS (SEQ ID NO: 8) |
| GST-PWWP-In- | MSPILGYWKIKGLVQPTRLLLEYLEEKYEEHLYERDEGDKWRNKKFELGLEFPNLPY |
| Chromo | YIDGDVKLTQSMAIIRYIADKHNMLGGCPKERAEISMLEGAVLDIRYGVSRIAYSKDF |
| | ETLKVDFLSKLPEMLKMFEDRLCHKTYLNGDHVTHPDFMLYDALDVVLYMDPMCL |
| | DAFPKLVCFKKRIEAIPQIDKYLKSSKYIAWPLQGWQATFGGGDHPPKSDLEVLFQG |
| | PLGSPGILGSEYEDGRGFGIGELVWGKLRGFSWWPGRIVSWWMTGRSRAAEGT |
| | RWVMWFGDGKFSVVCVEKLMPLSSFCSAFHQATYNKQPMYRKAIYEVLQVAS |
| | SRAGKLFPACHDSDESDSGKAVEVQNKQMIEWALGGFQPSGPKGLEPPLERPHR |
| | DVFEVEKILDMKTEGGKVLYKVRWKGYTSDDDTWEPEIHLEDCKEVLLEFRK |
| | KIAEPGSTRAAAS (SEQ ID NO: 9) |
| GST-PWWP- | MSPILGYWKIKGLVQPTRLLLEYLEEKYEEHLYERDEGDKWRNKKFELGLEFPNLPY |
| Chromo | YIDGDVKLTQSMAIIRYIADKHNMLGGCPKERAEISMLEGAVLDIRYGVSRIAYSKDF |
| | ETLKVDFLSKLPEMLKMFEDRLCHKTYLNGDHVTHPDFMLYDALDVVLYMDPMCL |
| | DAFPKLVCFKKRIEAIPQIDKYLKSSKYIAWPLQGWQATFGGGDHPPKSDLEVLFQG |
| | PLGSPGILGSEYEDGRGFGIGELVWGKLRGFSWWPGRIVSWWMTGRSRAAEGT |
| | RWVMWFGDGKFSVVCVEKLMPLSSFCSAFHQATYNKQPMYRKAIYEVLQVAS |
| | SRAGKLFPACHDSDESDSGKAVEVQNKQMIEWALGGFQPSGPKGLEPPDVFEV |
| | EKILDMKTEGGKVLYKVRWKGYTSDDDTWEPEIHLEDCKEVLLEFRKKIAEPG |
| | STRAAAS (SEQ ID NO: 10) |
| GST-PWWP- | MSPILGYWKIKGLVQPTRLLLEYLEEKYEEHLYERDEGDKWRNKKFELGLEFPNLPY |
| lnGS-Chromo | YIDGDVKLTQSMAIIRYIADKHNMLGGCPKERAEISMLEGAVLDIRYGVSRIAYSKDF |
| | ETLKVDFLSKLPEMLKMFEDRLCHKTYLNGDHVTHPDFMLYDALDVVLYMDPMCL |
| | DAFPKLVCFKKRIEAIPQIDKYLKSSKYIAWPLQGWQATFGGGDHPPKSDLEVLFQG |
| | PLGSPGILGSEYEDGRGFGIGELVWGKLRGFSWWPGRIVSWWMTGRSRAAEGT |
| | RWVMWFGDGKFSVVCVEKLMPLSSFCSAFHQATYNKQPMYRKAIYEVLQVAS |
| | SRAGKLFPACHDSDESDSGKAVEVQNKQMIEWALGGFQPSGPKGLEPPLERPHR |
| | GGGGSDVFEVEKILDMKTEGGKVLYKVRWKGYTSDDDTWEPEIHLEDCKEVLL |
| | EFRKKIAEPGSTRAAAS (SEQ ID NO: 11) |
| PWWP-In- | MGSPGILGSEYEDGRGFGIGELVWGKLRGFSWWPGRIVSWWMTGRSRAAEGT |
| Chromo | RWVMWFGDGKFSVVCVEKLMPLSSFCSAFHQATYNKQPMYRKAIYEVLQVAS |
| | SRAGKLFPACHDSDESDSGKAVEVQNKQMIEWALGGFQPSGPKGLEPPLERPHR |
| | DVFEVEKILDMKTEGGKVLYKVRWKGYTSDDDTWEPEIHLEDCKEVLLEFRK |
| | KIAEPGSTRAAAS (SEQ ID NO: 12) |
| PWWP-Chromo | MGSPGILGSEYEDGRGFGIGELVWGKLRGFSWWPGRIVSWWMTGRSRAAEGT |
| | RWVMWFGDGKFSVVCVEKLMPLSSFCSAFHQATYNKQPMYRKAIYEVLQVAS |
| | SRAGKLFPACHDSDESDSGKAVEVQNKQMIEWALGGFQPSGPKGLEPPDVFEV |
| | EKILDMKTEGGKVLYKVRWKGYTSDDDTWEPEIHLEDCKEVLLEFRKKIAEPG |
| | STRAAAS (SEQ ID NO: 13) |
| PWWP-InGS- | MGSPGILGSEYEDGRGFGIGELVWGKLRGFSWWPGRIVSWWMTGRSRAAEGT |
| Chromo | RWVMWFGDGKFSVVCVEKLMPLSSFCSAFHQATYNKQPMYRKAIYEVLQVAS |
| | SRAGKLFPACHDSDESDSGKAVEVQNKQMIEWALGGFQPSGPKGLEPPLERPHR |
| | GGGGSDVFEVEKILDMKTEGGKVLYKVRWKGYTSDDDTWEPEIHLEDCKEVLL |
| | EFRKKIAEPGSTRAAAS (SEQ ID NO: 14) |
| 7xHIS-MBP- | MKHHHHHHHGAAGTSLYKKAGMKIEEGKLVIWINGDKGYNGLAEVGKKFEKDTGI |
| 1xBAH | KVTVEHPDKLEEKFPQVAATGDGPDIIFWAHDRFGGYAQSGLLAEITPDKAFQDKLY |
| | PFTWDAVRYNGKLIAYPIAVEALSLIYNKDLLPNPPKTWEEIPALDKELKAKGKSAL |
| | MFNLQEPYFTWPLIAADGGYAFKYENGKYDIKDVGVDNAGAKAGLTFLVDLIKNK |
| | HMNADTDYSIAEAAFNKGETAMTINGPWAWSNIDTSKVNYGVTVLPTFKGQPSKPF |
| | VGVLSAGINAASPNKELAKEFLENYLLTDEGLEAVNKDKPLGAVALKSYEEELAKD |
| | PRIAATMENAQKGEIMPNIPQMSAFWYAVRTAVINAASGRQTVDEALKDAQTNSSS |
| | NNNNNNNNNNLGENLYFQGSAHYPTRLTTRKTYSWVGRPLLDRKLHYQTYREM |
| | CVKTEGCSTEIHIQIGQFVLIEGDDDENPYVAKLLELFEDDSDPPPKKRARVQWF |
| | VRFCEVPACKRHLLGRKPGAQEIFWYDYPACDSNINAETIIGLVRVIPLAPKDVV |
| | PTNLKNEKTLFVKLSWNEKKFRPLSSELFAELNKPQESAAK (SEQ ID NO: 15) |
| 7xHIS-MBP- | MKHHHHHHHGAAGTSLYKKAGMKIEEGKLVIWINGDKGYNGLAEVGKKFEKDTGI |
| 2xBAH | KVTVEHPDKLEEKFPQVAATGDGPDIIFWAHDRFGGYAQSGLLAEITPDKAFQDKLY |
| | PFTWDAVRYNGKLIAYPIAVEALSLIYNKDLLPNPPKTWEEIPALDKELKAKGKSAL |
| | MFNLQEPYFTWPLIAADGGYAFKYENGKYDIKDVGVDNAGAKAGLTFLVDLIKNK |
| | HMNADTDYSIAEAAFNKGETAMTINGPWAWSNIDTSKVNYGVTVLPTFKGQPSKPF |
| | VGVLSAGINAASPNKELAKEFLENYLLTDEGLEAVNKDKPLGAVALKSYEEELAKD |
| | PRIAATMENAQKGEIMPNIPQMSAFWYAVRTAVINAASGRQTVDEALKDAQTNSSS |
| | NNNNNNNNNNLGENLYFQGSAHYPTRLTTRKTYSWVGRPLLDRKLHYQTYREM |
| | CVKTEGCSTEIHIQIGQFVLIEGDDDENPYVAKLLELFEDDSDPPPKKRARVQWF |
| | VRFCEVPACKRHLLGRKPGAQEIFWYDYPACDSNINAETIIGLVRVIPLAPKDVV |
| | PTNLKNEKTLFVKLSWNEKKFRPLSSELFAELNKPQESAAKGGGGSGGGGSAHY |
| | PTRLTTRKTYSWVGRPLLDRKLHYQTYREMCVKTEGCSTEIHIQIGQFVLIEGD |
| | DDENPYVAKLLELFEDDSDPPPKKRARVQWFVRFCEVPACKRHLLGRKPGAQE |
| | IFWYDYPACDSNINAETIIGLVRVIPLAPKDVVPTNLKNEKTLFVKLSWNEKKFR |
| | PLSSELFAELNKPQESAAK (SEQ ID NO: 16) |
| 7xHIS-MBP- | MKHHHHHHHGAAGTSLYKKAGMKIEEGKLVIWINGDKGYNGLAEVGKKFEKDTGI |
| 3xBAH | KVTVEHPDKLEEKFPQVAATGDGPDIIFWAHDRFGGYAQSGLLAEITPDKAFQDKLY |
| | PFTWDAVRYNGKLIAYPIAVEALSLIYNKDLLPNPPKTWEEIPALDKELKAKGKSAL |
| | MFNLQEPYFTWPLIAADGGYAFKYENGKYDIKDVGVDNAGAKAGLTFLVDLIKNK |
| | HMNADTDYSIAEAAFNKGETAMTINGPWAWSNIDTSKVNYGVTVLPTFKGQPSKPF |
| | VGVLSAGINAASPNKELAKEFLENYLLTDEGLEAVNKDKPLGAVALKSYEEELAKD |
| | PRIAATMENAQKGEIMPNIPQMSAFWYAVRTAVINAASGRQTVDEALKDAQTNSSS |
| | NNNNNNNNNNLGENLYFQGSAHYPTRLTTRKTYSWVGRPLLDRKLHYQTYREM |
| | CVKTEGCSTEIHIQIGQFVLIEGDDDENPYVAKLLELFEDDSDPPPKKRARVQWF |
| | VRFCEVPACKRHLLGRKPGAQEIFWYDYPACDSNINAETIIGLVRVIPLAPKDVV |
| | PTNLKNEKTLFVKLSWNEKKFRPLSSELFAELNKPQESAAKGGGGSGGGGSAHY |
| | PTRLTTRKTYSWVGRPLLDRKLHYQTYREMCVKTEGCSTEIHIQIGQFVLIEGD |
| | DDENPYVAKLLELFEDDSDPPPKKRARVQWFVRFCEVPACKRHLLGRKPGAQE |
| | IFWYDYPACDSNINAETIIGLVRVIPLAPKDVVPTNLKNEKTLFVKLSWNEKKFR |
| | PLSSELFAELNKPQESAAKGGGGSGGGGSAHYPTRLTTRKTYSWVGRPLLDRKL |
| | HYQTYREMCVKTEGCSTEIHIQIGQFVLIEGDDDENPYVAKLLELFEDDSDPPPK |
| | KRARVQWFVRFCEVPACKRHLLGRKPGAQEIFWYDYPACDSNINAETIIGLVRV |
| | IPLAPKDVVPTNLKNEKTLFVKLSWNEKKFRPLSSELFAELNKPQESAAK (SEQ |
| | ID NO: 17) |
| MBP-1xCBX1 | MGSSHHHHHHGTKTEEGKLVIWINGDKGYNGLAEVGKKFEKDTGIKVTVEHPDKL |
| | EEKFPQVAATGDGPDIIFWAHDRFGGYAQSGLLAEITPDKAFQDKLYPFTWDAVRY |
| | NGKLIAYPIAVEALSLIYNKDLLPNPPKTWEEIPALDKELKAKGKSALMFNLQEPYFT |
| | WPLIAADGGYAFKYENGKYDIKDVGVDNAGAKAGLTFLVDLIKNKHMNADTDYSI |
| | AEAAFNKGETAMTINGPWAWSNIDTSKVNYGVTVLPTFKGQPSKPFVGVLSAGINA |
| | ASPNKELAKEFLENYLLTDEGLEAVNKDKPLGAVALKSYEEELAKDPRIAATMENA |
| | QKGEIMPNIPQMSAFWYAVRTAVINAASGRQTVDEALKDAQTGTDYDIPTTENLYF |
| | QGHMEEEEEEYVVEKVLDRRVVKGKVEYLLKWKGFSDEDNTWEPEENLDCPD |
| | LIAEFLQSQKT (SEQ ID NO: 41) |
| MBP-2xCBX1 | MGSSHHHHHHGTKTEEGKLVIWINGDKGYNGLAEVGKKFEKDTGIKVTVEHPDKL |
| | EEKFPQVAATGDGPDIIFWAHDRFGGYAQSGLLAEITPDKAFQDKLYPFTWDAVRY |
| | NGKLIAYPIAVEALSLIYNKDLLPNPPKTWEEIPALDKELKAKGKSALMFNLQEPYFT |
| | WPLIAADGGYAFKYENGKYDIKDVGVDNAGAKAGLTFLVDLIKNKHMNADTDYSI |
| | AEAAFNKGETAMTINGPWAWSNIDTSKVNYGVTVLPTFKGQPSKPFVGVLSAGINA |
| | ASPNKELAKEFLENYLLTDEGLEAVNKDKPLGAVALKSYEEELAKDPRIAATMENA |
| | QKGEIMPNIPQMSAFWYAVRTAVINAASGRQTVDEALKDAQTGTDYDIPTTENLYF |
| | QGHMEEEEEEYVVEKVLDRRVVKGKVEYLLKWKGFSDEDNTWEPEENLDCPD |
| | LIAEFLQSQKTGGGGSGGGGSEEEEEEYVVEKVLDRRVVKGKVEYLLKWKGFS |
| | DEDNTWEPEENLDCPDLIAEFLQSQKT (SEQ ID NO: 42) |
| MBP-3xCBX1 | MGSSHHHHHHGTKTEEGKLVIWINGDKGYNGLAEVGKKFEKDTGIKVTVEHPDKLE |
| | EKFPQVAATGDGPDIIFWAHDRFGGYAQSGLLAEITPDKAFQDKLYPFTWDAVRYN |
| | GKLIAYPIAVEALSLIYNKDLLPNPPKTWEEIPALDKELKAKGKSALMFNLQEPYFTW |
| | PLIAADGGYAFKYENGKYDIKDVGVDNAGAKAGLTFLVDLIKNKHMNADTDYSIAE |
| | AAFNKGETAMTINGPWAWSNIDTSKVNYGVTVLPTFKGQPSKPFVGVLSAGINAASP |
| | NKELAKEFLENYLLTDEGLEAVNKDKPLGAVALKSYEEELAKDPRIAATMENAQKG |
| | EIMPNIPQMSAFWYAVRTAVINAASGRQTVDEALKDAQTGTDYDIPTTENLYFQGH |
| | MEEEEEEYVVEKVLDRRVVKGKVEYLLKWKGFSDEDNTWEPEENLDCPDLIAE |
| | FLQSQKTGGGGSGGGGSEEEEEEYVVEKVLDRRVVKGKVEYLLKWKGFSDEDN |
| | TWEPEENLDCPDLIAEFLQSQKTGGGGSGGGGSEEEEEEYVVEKVLDRRVVKGK |
| | VEYLLKWKGFSDEDNTWEPEENLDCPDLIAEFLQSQKT (SEQ ID NO: 43) |
| MBP-1xCBX1 | MGSSHHHHHHGTKTEEGKLVIWINGDKGYNGLAEVGKKFEKDTGIKVTVEHPDKLE |
| eR | EKFPQVAATGDGPDIIFWAHDRFGGYAQSGLLAEITPDKAFQDKLYPFTWDAVRYN |
| | GKLIAYPIAVEALSLIYNKDLLPNPPKTWEEIPALDKELKAKGKSALMFNLQEPYFTW |
| | PLIAADGGYAFKYENGKYDIKDVGVDNAGAKAGLTFLVDLIKNKHMNADTDYSIAE |
| | AAFNKGETAMTINGPWAWSNIDTSKVNYGVTVLPTFKGQPSKPFVGVLSAGINAASP |
| | NKELAKEFLENYLLTDEGLEAVNKDKPLGAVALKSYEEELAKDPRIAATMENAQKG |
| | EIMPNIPQMSAFWYAVRTAVINAASGRQTVDEALKDAQTGTDYDIPTTENLYFQGH |
| | MEEEEEEYVVEKVLDRRVVKGKVEYLLKWAGFSDEDNTWEPEENLFCPDLIAE |
| | FLQSQK (SEQ ID NO: 44) |
| MBP-2xCBX1 | MGSSHHHHHHGTKTEEGKLVIWINGDKGYNGLAEVGKKFEKDTGIKVTVEHPDKLE |
| eR | EKFPQVAATGDGPDIIFWAHDRFGGYAQSGLLAEITPDKAFQDKLYPFTWDAVRYN |
| | GKLIAYPIAVEALSLIYNKDLLPNPPKTWEEIPALDKELKAKGKSALMFNLQEPYFTW |
| | PLIAADGGYAFKYENGKYDIKDVGVDNAGAKAGLTFLVDLIKNKHMNADTDYSIAE |
| | AAFNKGETAMTINGPWAWSNIDTSKVNYGVTVLPTFKGQPSKPFVGVLSAGINAASP |
| | NKELAKEFLENYLLTDEGLEAVNKDKPLGAVALKSYEEELAKDPRIAATMENAQKG |
| | EIMPNIPQMSAFWYAVRTAVINAASGRQTVDEALKDAQTGTDYDIPTTENLYFQGH |
| | MEEEEEEYVVEKVLDRRVVKGKVEYLLKWAGFSDEDNTWEPEENLFCPDLIAE |
| | FLQSQKTGGGGSGGGGSEEEEEEYVVEKVLDRRVVKGKVEYLLKWAGFSDEDN |
| | TWEPEENLFCPDLIAEFLQSQKT (SEQ ID NO: 45) |
| MBP-3xCBX1 | MGSSHHHHHHGTKTEEGKLVIWINGDKGYNGLAEVGKKFEKDTGIKVTVEHPDKLE |
| eR | EKFPQVAATGDGPDIIFWAHDRFGGYAQSGLLAEITPDKAFQDKLYPFTWDAVRYN |
| | GKLIAYPIAVEALSLIYNKDLLPNPPKTWEEIPALDKELKAKGKSALMFNLQEPYFTW |
| | PLIAADGGYAFKYENGKYDIKDVGVDNAGAKAGLTFLVDLIKNKHMNADTDYSIAE |
| | AAFNKGETAMTINGPWAWSNIDTSKVNYGVTVLPTFKGQPSKPFVGVLSAGINAASP |
| | NKELAKEFLENYLLTDEGLEAVNKDKPLGAVALKSYEEELAKDPRIAATMENAQKG |
| | EIMPNIPQMSAFWYAVRTAVINAASGRQTVDEALKDAQTGTDYDIPTTENLYFQGH |
| | MEEEEEEYVVEKVLDRRVVKGKVEYLLKWAGFSDEDNTWEPEENLFCPDLIAE |
| | FLQSQKTGGGGSGGGGSEEEEEEYVVEKVLDRRVVKGKVEYLLKWAGFSDEDN |
| | TWEPEENLFCPDLIAEFLQSQKTGGGGSGGGGSEEEEEEYVVEKVLDRRVVKGK |
| | VEYLLKWAGFSDEDNTWEPEENLFCPDLIAEFLQSQKT (SEQ ID NO: 46) |
| GST-2XLANA- | MSPILGYWKIKGLVQPTRLLLEYLEEKYEEHLYERDEGDKWRNKKFELGLEFPNLPY |
| 2xGS- | YIDGDVKLTQSMAIIRYIADKHNMLGGCPKERAEISMLEGAVLDIRYGVSRIAYSKDF |
| MeCP2 6xHIS | ETLKVDFLSKLPEMLKMFEDRLCHKTYLNGDHVTHPDFMLYDALDVVLYMDPMCL |
| | DAFPKLVCFKKRIEAIPQIDKYLKSSKYIAWPLQGWQATFGGGDHPPKSDLEVLFQG |
| | PLGSIEENLYFQSMAPPGMRLRSGRSTGAPLTRGSGGGGSMAPPGMRLRSGRST |
| | GAPLTRGSGGGGSGGGGSLPATGGASASPKQRRSIIRDRGPMYDDPTLPEGWTR |
| | KLKQRKSGRSAGKYDVYLINPQGKAFRSKVELIAYFEKVGDTSLDPNDFDFTVT |
| | GRGSPSRREGGHHHHHH* (SEQ ID NO: 18) |
| GST-2XLANA- | MSPILGYWKIKGLVQPTRLLLEYLEEKYEEHLYERDEGDKWRNKKFELGLEFPNLPY |
| 2xGS- | YIDGDVKLTQSMAIIRYIADKHNMLGGCPKERAEISMLEGAVLDIRYGVSRIAYSKDF |
| MeCP2(Y123F) | ETLKVDFLSKLPEMLKMFEDRLCHKTYLNGDHVTHPDFMLYDALDVVLYMDPMCL |
| 6xHIS | DAFPKLVCFKKRIEAIPQIDKYLKSSKYIAWPLQGWQATFGGGDHPPKSDLEVLFQG |
| | PLGSIEENLYFQSMAPPGMRLRSGRSTGAPLTRGSGGGGSMAPPGMRLRSGRST |
| | GAPLTRGSGGGGSGGGGSLPATGGASASPKQRRSIIRDRGPMYDDPTLPEGWTR |
| | KLKQRKSGRSAGKYDVFLINPQGKAFRSKVELIAYFEKVGDTSLDPNDFDFTVT |
| | GRGSPSRREGGHHHHHH (SEQ ID NO: 19) |
| GST-2XLANA- | MSPILGYWKIKGLVQPTRLLLEYLEEKYEEHLYERDEGDKWRNKKFELGLEFPNLPY |
| 2xGS- | YIDGDVKLTQSMAIIRYIADKHNMLGGCPKERAEISMLEGAVLDIRYGVSRIAYSKDF |
| MeCP2(T158S)_ | ETLKVDFLSKLPEMLKMFEDRLCHKTYLNGDHVTHPDFMLYDALDVVLYMDPMCL |
| 6xHIS | DAFPKLVCFKKRIEAIPQIDKYLKSSKYIAWPLQGWQATFGGGDHPPKSDLEVLFQG |
| | PLGSENLYFQGGASASPKQRRSIIRDRGPMYDDPTLPEGWTRKLKQRKSGRSAG |
| | KYDVYLINPQGKAFRSKVELIAYFEKVGDTSLDPNDFDFSVTGRGSPSRREHHHH |
| | HH* (SEQ ID NO: 20) |
| GST-2XLANA- | MSPILGYWKIKGLVQPTRLLLEYLEEKYEEHLYERDEGDKWRNKKFELGLEFPNLPY |
| 2xGS- | YIDGDVKLTQSMAIIRYIADKHNMLGGCPKERAEISMLEGAVLDIRYGVSRIAYSKDF |
| MeCP2(Y123F | ETLKVDFLSKLPEMLKMFEDRLCHKTYLNGDHVTHPDFMLYDALDVVLYMDPMCL |
| T158S) 6xHIS | DAFPKLVCFKKRIEAIPQIDKYLKSSKYIAWPLQGWQATFGGGDHPPKSDLEVLFQG |
| | PLGSENLYFQGGASASPKQRRSIIRDRGPMYDDPTLPEGWTRKLKQRKSGRSAG |
| | KYDVFLINPQGKAFRSKVELIAYFEKVGDTSLDPNDFDFSVTGRGSPSRREHHHH |
| | HH* (SEQ ID NO: 21) |
| GST-2XLANA- | MSPILGYWKIKGLVQPTRLLLEYLEEKYEEHLYERDEGDKWRNKKFELGLEFPNLPY |
| MeCP2 6xHIS | YIDGDVKLTQSMAIIRYIADKHNMLGGCPKERAEISMLEGAVLDIRYGVSRIAYSKDF |
| | ETLKVDFLSKLPEMLKMFEDRLCHKTYLNGDHVTHPDFMLYDALDVVLYMDPMCL |
| | DAFPKLVCFKKRIEAIPQIDKYLKSSKYIAWPLQGWQATFGGGDHPPKSDLEVLFQG |
| | PLGSIEENLYFQSMAPPGMRLRSGRSTGAPLTRGSGGGGSMAPPGMRLRSGRST |
| | GAPLTRGSASASPKQRRSIIRDRGPMYDDPTLPEGWTRKLKQRKSGRSAGKYD |
| | VYLINPQGKAFRSKVELIAYFEKVGDTSLDPNDFDFTVTGRGSPSRREGGHHHHH |
| | H* (SEQ ID NO: 22) |
| GST-2XLANA- | MSPILGYWKIKGLVQPTRLLLEYLEEKYEEHLYERDEGDKWRNKKFELGLEFPNLPY |
| 1xGS- | YIDGDVKLTQSMAIIRYIADKHNMLGGCPKERAEISMLEGAVLDIRYGVSRIAYSKDF |
| MeCP2 6xHIS | ETLKVDFLSKLPEMLKMFEDRLCHKTYLNGDHVTHPDFMLYDALDVVLYMDPMCL |
| | DAFPKLVCFKKRIEAIPQIDKYLKSSKYIAWPLQGWQATFGGGDHPPKSDLEVLFQG |
| | PLGSIEENLYFQSMAPPGMRLRSGRSTGAPLTRGSGGGGSMAPPGMRLRSGRST |
| | GAPLTRGSGGGGSLPATGGASASPKQRRSIIRDRGPMYDDPTLPEGWTRKLKQR |
| | KSGRSAGKYDVYLINPQGKAFRSKVELIAYFEKVGDTSLDPNDFDFTVTGRGSPS |
| | RREGGHHHHHH* (SEQ ID NO: 23) |
| GST-2XLANA- | MSPILGYWKIKGLVQPTRLLLEYLEEKYEEHLYERDEGDKWRNKKFELGLEFPNLPY |
| 3xGS- | YIDGDVKLTQSMAIIRYIADKHNMLGGCPKERAEISMLEGAVLDIRYGVSRIAYSKDF |
| MeCP2 6xHIS | ETLKVDFLSKLPEMLKMFEDRLCHKTYLNGDHVTHPDFMLYDALDVVLYMDPMCL |
| | DAFPKLVCFKKRIEAIPQIDKYLKSSKYIAWPLQGWQATFGGGDHPPKSDLEVLFQG |
| | PLGSIEENLYFQSMAPPGMRLRSGRSTGAPLTRGSGGGGSMAPPGMRLRSGRST |
| | GAPLTRGSGGGGSGGGGSGGGGSLPATGGASASPKQRRSIIRDRGPMYDDPTLPE |
| | GWTRKLKQRKSGRSAGKYDVYLINPQGKAFRSKVELIAYFEKVGDTSLDPNDF |
| | DFTVTGRGSPSRREGGHHHHHH* (SEQ ID NO: 24) |
| GST-2XLANA- | MSPILGYWKIKGLVQPTRLLLEYLEEKYEEHLYERDEGDKWRNKKFELGLEFPNLPY |
| MeCP2 GSflex | YIDGDVKLTQSMAIIRYIADKHNMLGGCPKERAEISMLEGAVLDIRYGVSRIAYSKDF |
| | ETLKVDFLSKLPEMLKMFEDRLCHKTYLNGDHVTHPDFMLYDALDVVLYMDPMCL |
| | DAFPKLVCFKKRIEAIPQIDKYLKSSKYIAWPLQGWQATFGGGDHPPKSDLEVLFQG |
| | PLGSIEENLYFQSMAPPGMRLRSGRSTGAPLTRGSGGGGSMAPPGMRLRSGRST |
| | GAPLTRGSGSAGSAAGSGEFKLPATGGASASPKQRRSIIRDRGPMYDDPTLPEGW |
| | TRKLKQRKSGRSAGKYDVYLINPQGKAFRSKVELIAYFEKVGDTSLDPNDFDFT |
| | VTGRGSPSRREGGHHHHHH* (SEQ ID NO: 25) |
| GST-2XLANA- | MSPILGYWKIKGLVQPTRLLLEYLEEKYEEHLYERDEGDKWRNKKFELGLEFPNLPY |
| MeCP2 flex1 | YIDGDVKLTQSMAIIRYIADKHNMLGGCPKERAEISMLEGAVLDIRYGVSRIAYSKDF |
| | ETLKVDFLSKLPEMLKMFEDRLCHKTYLNGDHVTHPDFMLYDALDVVLYMDPMCL |
| | DAFPKLVCFKKRIEAIPQIDKYLKSSKYIAWPLQGWQATFGGGDHPPKSDLEVLFQG |
| | PLGSIEENLYFQSMAPPGMRLRSGRSTGAPLTRGSGGGGSMAPPGMRLRSGRST |
| | GAPLTRGSKESGSVSSEQLAQFRSLDKLPATGGASASPKQRRSIIRDRGPMYDDPT |
| | LPEGWTRKLKQRKSGRSAGKYDVYLINPQGKAFRSKVELIAYFEKVGDTSLDP |
| | NDFDFTVTGRGSPSRREGGHHHHHH* (SEQ ID NO: 26) |
| GST-2XLANA- | MSPILGYWKIKGLVQPTRLLLEYLEEKYEEHLYERDEGDKWRNKKFELGLEFPNLPY |
| MeCP2 flex2 | YIDGDVKLTQSMAIIRYIADKHNMLGGCPKERAEISMLEGAVLDIRYGVSRIAYSKDF |
| | ETLKVDFLSKLPEMLKMFEDRLCHKTYLNGDHVTHPDFMLYDALDVVLYMDPMCL |
| | DAFPKLVCFKKRIEAIPQIDKYLKSSKYIAWPLQGWQATFGGGDHPPKSDLEVLFQG |
| | PLGSIEENLYFQSMAPPGMRLRSGRSTGAPLTRGSGGGGSMAPPGMRLRSGRST |
| | GAPLTRGSEGKSSGSGSESKSTKLPATGGASASPKQRRSIIRDRGPMYDDPTLPEG |
| | WTRKLKQRKSGRSAGKYDVYLINPQGKAFRSKVELIAYFEKVGDTSLDPNDFDF |
| | TVTGRGSPSRREGGHHHHHH* (SEQ ID NO: 27) |
| GST-2XGS_ | MSPILGYWKIKGLVQPTRLLLEYLEEKYEEHLYERDEGDKWRNKKFELGLEFPNLPY |
| 2XLANA- | YIDGDVKLTQSMAIIRYIADKHNMLGGCPKERAEISMLEGAVLDIRYGVSRIAYSKDF |
| 2xGS- | ETLKVDFLSKLPEMLKMFEDRLCHKTYLNGDHVTHPDFMLYDALDVVLYMDPMCL |
| MeCP2 6xHIS | DAFPKLVCFKKRIEAIPQIDKYLKSSKYIAWPLQGWQATFGGGDHPPKSDLEVLFQG |
| | PLGSIEENLYFQSGSGGSGSGGMAPPGMRLRSGRSTGAPLTRGSGGGGSMAPPG |
| | MRLRSGRSTGAPLTRGSGGGGSGGGGSLPATGGASASPKQRRSIIRDRGPMYDDP |
| | TLPEGWTRKLKQRKSGRSAGKYDVYLINPQGKAFRSKVELIAYFEKVGDTSLDP |
| | NDFDFTVTGRGSPSRREGGHHHHHH* (SEQ ID NO: 28) |
| GST-hCMV- | MSPILGYWKIKGLVQPTRLLLEYLEEKYEEHLYERDEGDKWRNKKFELGLEFPNLPY |
| MeCP2 | YIDGDVKLTQSMAIIRYIADKHNMLGGCPKERAEISMLEGAVLDIRYGVSRIAYSKDF |
| | ETLKVDFLSKLPEMLKMFEDRLCHKTYLNGDHVTHPDFMLYDALDVVLYMDPMCL |
| | DAFPKLVCFKKRIEAIPQIDKYLKSSKYIAWPLQGWQATFGGGDHPPKSDLEVLFQG |
| | PLGSIEENLYFQSGSGGSGSGGKSTHPMVTRSKASASPKQRRSIIRDRGPMYDDPTL |
| | PEGWTRKLKQRKSGRSAGKYDVYLINPQGKAFRSKVELIAYFEKVGDTSLDPN |
| | DFDFTVTGRGSPSRREGGHHHHHH* (SEQ ID NO: 29) |
| GST-hCMV- | MSPILGYWKIKGLVQPTRLLLEYLEEKYEEHLYERDEGDKWRNKKFELGLEFPNLPY |
| 2XGS-MeCP2 | YIDGDVKLTQSMAIIRYIADKHNMLGGCPKERAEISMLEGAVLDIRYGVSRIAYSKDF |
| | ETLKVDFLSKLPEMLKMFEDRLCHKTYLNGDHVTHPDFMLYDALDVVLYMDPMCL |
| | DAFPKLVCFKKRIEAIPQIDKYLKSSKYIAWPLQGWQATFGGGDHPPKSDLEVLFQG |
| | PLGSIEENLYFQSGSGGSGSGGKSTHPMVTRSKGGGGSGGGGSLPATGGASASPKQR |
| | RSIIRDRGPMYDDPTLPEGWTRKLKQRKSGRSAGKYDVYLINPQGKAFRSKVEL |
| | JAYFEKVGDTSLDPNDFDFTVTGRGSPSRREGGHHHHHH* (SEQ ID NO: 30) |
| GST-Coiled | MSPILGYWKIKGLVQPTRLLLEYLEEKYEEHLYERDEGDKWRNKKFELGLEFPNLPY |
| Coil-LANA- | YIDGDVKLTQSMAIIRYIADKHNMLGGCPKERAEISMLEGAVLDIRYGVSRIAYSKDF |
| MeCP2 | ETLKVDFLSKLPEMLKMFEDRLCHKTYLNGDHVTHPDFMLYDALDVVLYMDPMCL |
| | DAFPKLVCFKKRIEAIPQIDKYLKSSKYIAWPLQGWQATFGGGDHPPKSDLEVLFQG |
| | PLGSIEENLYFQSGSGGSGSGGEELRRRLEELRRRRGGGGMRLRSGRSTGGGGLE |
| | EEIRRLEEEIRREASASPKQRRSIIRDRGPMYDDPTLPEGWTRKLKQRKSGRSAG |
| | KYDVYLINPQGKAFRSKVELIAYFEKVGDTSLDPNDFDFTVTGRGSPSRREGGH |
| | HHHHH* (SEQ ID NO: 31) |
| GST-Coiled | MSPILGYWKIKGLVQPTRLLLEYLEEKYEEHLYERDEGDKWRNKKFELGLEFPNLPY |
| Coil-LANA- | YIDGDVKLTQSMAIIRYIADKHNMLGGCPKERAEISMLEGAVLDIRYGVSRIAYSKDF |
| MeCP2 w/ | ETLKVDFLSKLPEMLKMFEDRLCHKTYLNGDHVTHPDFMLYDALDVVLYMDPMCL |
| linker | DAFPKLVCFKKRIEAIPQIDKYLKSSKYIAWPLQGWQATFGGGDHPPKSDLEVLFQG |
| | PLGSIEENLYFQSGSGGSGSGGEELRRRLEELRRRRGMRLRSGRSTGLEEEIRRLE |
| | EEIRREASASPKQRRSIIRDRGPMYDDPTLPEGWTRKLKQRKSGRSAGKYDVYL |
| | INPQGKAFRSKVELIAYFEKVGDTSLDPNDFDFTVTGRGSPSRREGGHHHHHH* |
| | (SEQ ID NO: 32) |
| GST-Coiled | MSPILGYWKIKGLVQPTRLLLEYLEEKYEEHLYERDEGDKWRNKKFELGLEFPNLPY |
| Coil-LANA- | YIDGDVKLTQSMAIIRYIADKHNMLGGCPKERAEISMLEGAVLDIRYGVSRIAYSKDF |
| 2xGS-MeCP2 w/ | ETLKVDFLSKLPEMLKMFEDRLCHKTYLNGDHVTHPDFMLYDALDVVLYMDPMCL |
| linker | DAFPKLVCFKKRIEAIPQIDKYLKSSKYIAWPLQGWQATFGGGDHPPKSDLEVLFQG |
| | PLGSIEENLYFQSGSGGSGSGGEELRRRLEELRRRRGGGGMRLRSGRSTGGGGLE |
| | EEIRRLEEEIRREGGGGSGGGGSLPATGGASASPKQRRSIIRDRGPMYDDPTLPEG |
| | WTRKLKQRKSGRSAGKYDVYLINPQGKAFRSKVELIAYFEKVGDTSLDPNDFDF |
| | TVTGRGSPSRREGGHHHHHH* (SEQ ID NO: 33) |
| GST-Coiled | MSPILGYWKIKGLVQPTRLLLEYLEEKYEEHLYERDEGDKWRNKKFELGLEFPNLPY |
| Coil-LANA- | YIDGDVKLTQSMAIIRYIADKHNMLGGCPKERAEISMLEGAVLDIRYGVSRIAYSKDF |
| 2xGS-MeCP2 | ETLKVDFLSKLPEMLKMFEDRLCHKTYLNGDHVTHPDFMLYDALDVVLYMDPMCL |
| | DAFPKLVCFKKRIEAIPQIDKYLKSSKYIAWPLQGWQATFGGGDHPPKSDLEVLFQG |
| | PLGSIEENLYFQSGSGGSGSGGEELRRRLEELRRRRGMRLRSGRSTGLEEEIRRLE |
| | EEIRREGGGGSGGGGSLPATGGASASPKQRRSIIRDRGPMYDDPTLPEGWTRKLK |
| | QRKSGRSAGKYDVYLINPQGKAFRSKVELIAYFEKVGDTSLDPNDFDFTVTGRG |
| | SPSRREGGHHHHHH* (SEQ ID NO: 34) |
| GST-BPTF | MSPILGYWKIKGLVQPTRLLLEYLEEKYEEHLYERDEGDKWRNKKFELGLEFPNLP |
| (PHD-BD) | YYIDGDVKLTQSMAIIRYIADKHNMLGGCPKERAEISMLEGAVLDIRYGVSRIAYSK |
| | DFETLKVDFLSKLPEMLKMFEDRLCHKTYLNGDHVTHPDFMLYDALDVVLYMDPM |
| | CLDAFPKLVCFKKRIEAIPQIDKYLKSSKYIAWPLQGWQATFGGGDHPPKSDLEVLF |
| | QGPLGSPGIPDTKLYCICKTPYDESKFYIGCDRCQNWYHGRCVGILQSEAELIDE |
| | YVCPQCQSTEDAMTVLTPLTEKDYEGLKRVLRSLQAHKMAWPFLEPVDPNDAP |
| | DYYGVIKEPMDLATMEERVQRRYYEKLTEFVADMTKIFDNCRYYNPSDSPFYQ |
| | CAEVLESFFVQKLKGFKASRS (SEQ ID NO: 47) |
| GST-BPTF | MSPILGYWKIKGLVQPTRLLLEYLEEKYEEHLYERDEGDKWRNKKFELGLEFPNLPY |
| (PHD)-CBX7 | YIDGDVKLTQSMAIIRYIADKHNMLGGCPKERAEISMLEGAVLDIRYGVSRIAYSKDF |
| (CD) | ETLKVDFLSKLPEMLKMFEDRLCHKTYLNGDHVTHPDFMLYDALDVVLYMDPMCL |
| | DAFPKLVCFKKRIEAIPQIDKYLKSSKYIAWPLQGWQATFGGGDHPPKSDLEVLFQG |
| | PLGSENLYFQGGDTKLYCICKTPYDESKFYIGCDRCQNWYHGRCVGILQSEAELI |
| | DEYVCPQCQSTEDAMTVGGGGSMSKGEELFTGVVPILVELDGDVNGHKFSVSG |
| | EGEGDATYGKLTLKFICTTGKLPVPWPTLVTTFTYGVQCFSRYPDHMKRHDFF |
| | KSAMPEGYVQERTIFFKDDGNYKTRAEVKFEGDTLVNRIELKGIDFKEDGNILG |
| | HKLEYNYNSHNVYIMADKQKNGIKVNFKIRHNIEDGSVQLADHYQONTPIGDGP |
| | VLLPDNHYLSTQSALSKDPNEKRDHMVLLEFVTAAGITHGMDELYKGGGGSEQ |
| | VFAVESIRKKRVRKGKVEYLVKWKGWPPKYSTWEPEEHILDPRLVMAYEEKE |
| | ERDRASGY (SEQ ID NO: 35) |
| GST-1xTAF3 | MSPILGYWKIKGL VQPTRLLLEYLEEKYEEHLYERDEGDKWRNKKFELGLEFPNLPY |
| | YIDGDVKLTQSMAIIRYIADKHNMLGGCPKERAEISMLEGAVLDIRYGVSRIAYSKDF |
| | ETLKVDFLSKLPEMLKMFEDRLCHKTYLNGDHVTHPDFMLYDALDVVLYMDPMCL |
| | DAFPKLVCFKKRIEAIPQIDKYLKSSKYIAWPLQGWQATFGGGDHPPKSDLEVLFQG |
| | PLGSENLYFQGGVIRDEWGNQIWICPGCNKPDDGSPMIGCDDCDDWYHWPCVG |
| | IMTAPPEEMQWFCPKCANKKKDKKHKKRKHRAHGGGGSDYKDDDDK (SEQ ID |
| | NO: 36) |
| GST- | MSPILGYWKIKGLVQPTRLLLEYLEEKYEEHLYERDEGDKWRNKKFELGLEFPNLPY |
| 2xTAF3_GS | YIDGDVKLTQSMAIIRYIADKHNMLGGCPKERAEISMLEGAVLDIRYGVSRIAYSKDF |
| | ETLKVDFLSKLPEMLKMFEDRLCHKTYLNGDHVTHPDFMLYDALDVVLYMDPMCL |
| | DAFPKLVCFKKRIEAIPQIDKYLKSSKYIAWPLQGWQATFGGGDHPPKSDLEVLFQG |
| | PLGSENLYFQGGVIRDEWGNQIWICPGCNKPDDGSPMIGCDDCDDWYHWPCVG |
| | IMTAPPEEMQWFCPKCANKKKDKKHKKRKHRAHGGGGSGGGGSVIRDEWGN |
| | QIWICPGCNKPDDGSPMIGCDDCDDWYHWPCVGIMTAPPEEMQWFCPKCANK |
| | KKDKKHKKRKHRAHGGGGSDYKDDDDK (SEQ ID NO: 37) |
| GST- | MSPILGYWKIKGLVQPTRLLLEYLEEKYEEHLYERDEGDKWRNKKFELGLEFPNLPY |
| 3xTAF3_GS | YIDGDVKLTQSMAIIRYIADKHNMLGGCPKERAEISMLEGAVLDIRYGVSRIAYSKDF |
| | ETLKVDFLSKLPEMLKMFEDRLCHKTYLNGDHVTHPDFMLYDALDVVLYMDPMCL |
| | DAFPKLVCFKKRIEAIPQIDKYLKSSKYIAWPLQGWQATFGGGDHPPKSDLEVLFQG |
| | PLGSENLYFQGGVIRDEWGNQIWICPGCNKPDDGSPMIGCDDCDDWYHWPCVG |
| | IMTAPPEEMQWFCPKCANKKKDKKHKKRKHRAHGGGGSGGGGSVIRDEWGN |
| | QIWICPGCNKPDDGSPMIGCDDCDDWYHWPCVGIMTAPPEEMQWFCPKCANK |
| | KKDKKHKKRKHRAHGGGGSGGGGSVIRDEWGNQIWICPGCNKPDDGSPMIGC |
| | DDCDDWYHWPCVGIMTAPPEEMQWFCPKCANKKKDKKHKKRKHRAHGGGG |
| | SDYKDDDDK (SEQ ID NO: 38) |
| GST- | MSPILGYWKIKGLVQPTRLLLEYLEEKYEEHLYERDEGDKWRNKKFELGLEFPNLPY |
| 2xTAF3_NL | YIDGDVKLTQSMAIIRYIADKHNMLGGCPKERAEISMLEGAVLDIRYGVSRIAYSKDF |
| | ETLKVDFLSKLPEMLKMFEDRLCHKTYLNGDHVTHPDFMLYDALDVVLYMDPMCL |
| | DAFPKLVCFKKRIEAIPQIDKYLKSSKYIAWPLQGWQATFGGGDHPPKSDLEVLFQG |
| | PLGSENLYFQGGVIRDEWGNQIWICPGCNKPDDGSPMIGCDDCDDWYHWPCVG |
| | IMTAPPEEMQWFCPKCANKKKDKKHKKRKHRAHSVVTETVSTYVIRDEWGNQ |
| | IWICPGCNKPDDGSPMIGCDDCDDWYHWPCVGIMTAPPEEMQWFCPKCANKK |
| | KDKKHKKRKHRAHGGGGSDYKDDDDK (SEQ ID NO: 39) |
| GST- | MSPILGYWKIKGLVQPTRLLLEYLEEKYEEHLYERDEGDKWRNKKFELGLEFPNLPY |
| 3xTAF3_NL | YIDGDVKLTQSMAIIRYIADKHNMLGGCPKERAEISMLEGAVLDIRYGVSRIAYSKDF |
| | ETLKVDFLSKLPEMLKMFEDRLCHKTYLNGDHVTHPDFMLYDALDVVLYMDPMCL |
| | DAFPKLVCFKKRIEAIPQIDKYLKSSKYIAWPLQGWQATFGGGDHPPKSDLEVLFQG |
| | PLGSENLYFQGGVIRDEWGNQIWICPGCNKPDDGSPMIGCDDCDDWYHWPCVG |
| | IMTAPPEEMQWFCPKCANKKKDKKHKKRKHRAHSVVTETVSTYVIRDEWGNQ |
| | IWICPGCNKPDDGSPMIGCDDCDDWYHWPCVGIMTAPPEEMQWFCPKCANKK |
| | KDKKHKKRKHRAHSVVTETVSTYVIRDEWGNQIWICPGCNKPDDGSPMIGCDD |
| | CDDWYHWPCVGIMTAPPEEMQWFCPKCANKKKDKKHKKRKHRAHGGGGSD |
| | YKDDDDK (SEQ ID NO: 40) |

The foregoing examples are illustrative of the present invention, and are not to be construed as limiting thereof. Although the invention has been described in detail with reference to preferred embodiments, variations and modifications exist within the scope and spirit of the invention as described and defined in the following claims.

1. A method for screening a detection reagent for a chromatin element, comprising:
a. providing a panel of recombinant nucleosomes, each nucleosome comprising one or more chromatin elements recognized by one or more binding domains present in the detection reagent and/or one or more chromatin elements not recognized by the one or more binding domains present in the detection reagent, thereby providing both on-target and off-target recombinant nucleosomes; and
b. performing a genomics assay or binding assay with the panel of recombinant nucleosomes to identify the binding specificity and/or efficiency of the detection reagent.

2. The method of claim 1, wherein the detection reagent comprises a recombinant fusion protein and wherein the recombinant fusion protein detection reagent comprises one or more binding domains that recognize one or more of the chromatin elements in the panel of nucleosomes, and a label.

3. The method of claim 2, wherein the label is an epitope tag.

4. The method of claim 2, wherein the recombinant fusion protein comprises two or more of the same binding domains and optionally comprises a linker between each binding domain, or
wherein the recombinant fusion protein comprises two or more binding domains from the same binding protein and optionally comprises a linker between each binding domain, or wherein the fusion protein comprises two or more binding domains from different proteins, each domain recognizing one or more chromatin elements on a nucleosome, optionally comprising a linker between each binding domain.

5. (canceled)

6. The method of claim 1, wherein the genomic assay is for a single chromatin element.

7-8. (canceled)

9. The method of claim 1, wherein the nucleosomes are optionally DNA-barcoded and the assay is a genomic mapping assay or a binding assay, optionally wherein the assay maps the location of two or more chromatin targets on the same nucleosome or on adjacent nucleosomes.

10-12. (canceled)

13. The method of claim 1, wherein the chromatin elements are histone modifications, histone mutations, histone variants, DNA modifications, or proteins that directly or indirectly bind chromatin, or
wherein the chromatin elements comprise proteins that bind unmodified histones, optionally wherein the chromatin element is an acid binding patch, or
wherein the chromatin elements are comprised of both histone modifications and proteins that directly or indirectly bind chromatin.

14-15. (canceled)

16. The method of claim 1, wherein one or more of the binding domains is selected from bromodomains, chromodomains, PWWP domains, plant homeodomains (PHD), malignant brain tumor (MBT) domain, WD40 Repeat domain, Tandem Tudor and/or Tudor-like domains.

17. The method of claim 1, wherein the binding domains and linker are from a native protein, or
wherein the binding domains are from two or more proteins and the linker is non-native.

18. (canceled)

19. The method of claim 4, wherein the linker is flexible or rigid, optionally wherein the linker is comprised of (GGGGS)ₙ (SEQ ID NO:1), wherein n is between 1 and 4.

20. (canceled)

21. The method of claim 9, wherein the genomic mapping assay is performed on <10,000 cells, optionally <1,000-2,000 cells, optionally a single cell.

22-23. (canceled)

24. The method of claim 9 wherein the genomic mapping assay is CUT&RUN or CUT&Tag, or
wherein the genomic mapping assay uses an antibody to an epitope tag and the label on the fusion protein is the epitope tag, or
wherein the binding assay is a bead-based proximity assay or bead-based multiplex assay.

25-26. (canceled)

27. A recombinant fusion protein comprising two or more chromatin element binding domains, a label, and optionally a linker.

28. The recombinant fusion protein of claim 27, wherein the label is an epitope tag, optionally GST or MBP.

29. The recombinant fusion protein of claim 27, wherein at least one of the two or more binding domains are selected from bromodomains, chromodomains, PWWP domains, plant homeodomains (PHD), malignant brain tumor (MBT) domain, WD40 Repeat domain, Tandem Tudor and/or Tudor-like domains.

30. The recombinant fusion protein of claim 27, wherein the two or more binding domains are the same, or
wherein the two or more binding domains are different, or
wherein the two or more binding domains are from the same protein, or
wherein the two or more binding domains are from different proteins, or
wherein the two or more binding domains comprise a chromodomain and a PWWP domain, optionally wherein the chromodomain is from MPP8 and the PWWP domain is from DNMT3A, or
wherein the two or more binding domains comprise a bromodomain and a PHD domain, optionally, wherein the bromodomain and the PHD domain are from BPTF, or
wherein the two or more binding domains comprise two or more chromodomains, optionally
wherein the two or more binding domains comprise two or more chromodomains from CBX1, or
wherein the two or more binding domains comprise one or more PHD domains from TAF3.

31-41. (canceled)

42. The recombinant fusion protein of claim 27, wherein the linker is flexible or rigid, optionally wherein the linker is comprised of (GGGGS)ₙ (SEQ ID NO:1), wherein n is between 1 and 4.

43. (canceled)

44. The recombinant fusion protein of claim 27, wherein the protein comprises an amino acid sequence at least 80% sequence identical to any one of SEQ ID NOs: 7-47.

45. A genomic mapping assay method, wherein the method is carried out using the recombinant fusion protein of claim 27.

46-53. (canceled)

54. A kit comprising the recombinant fusion protein of claim 27.


SEQUENCE LISTING (47 sequences)

SEQ ID NO: 1 - protein, 20 aa, synthetic construct
  VARIANT 6..20: note = Residue may be present or absent
GGGGSGGGGSGGGGSGGGGS

SEQ ID NO: 2 - protein, 10 aa, synthetic construct
  VARIANT 5..10: note = Residue may be present or absent
GGGGGGGGGG

SEQ ID NO: 3 - protein, 20 aa, synthetic construct
  VARIANT 6..20: note = Residue may be present or absent
EAAAKEAAAKEAAAKEAAAK

SEQ ID NO: 4 - protein, 20 aa, synthetic construct
  VARIANT 3..20: note = Residue is present or absent
XPXPXPXPXPXPXPXPXPXP

SEQ ID NO: 5 - protein, 10 aa, synthetic construct
GGGGSGGGGS

SEQ ID NO: 6 - protein, 5 aa, synthetic construct
GGGGS

SEQ ID NO: 7 - protein, 380 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSDTKLYCICKTPYDESKFYIGCDRCQNWYHGRCVGILQSEAELIDEYVCPQCQSTEDAMTVLTPLTEKDYEGLKRVLRSLGGGGSDVFEVEKILDMKTEGGKVLYKVRWKGYTSDDDTWEPEIHLEDCKEVLLEFRKKIAEPGSTRAAAS

SEQ ID NO: 8 - protein, 375 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSDTKLYCICKTPYDESKFYIGCDRCQNWYHGRCVGILQSEAELIDEYVCPQCQSTEDAMTVLTPLTEKDYEGLKRVLRSLDVFEVEKILDMKTEGGKVLYKVRWKGYTSDDDTWEPEIHLEDCKEVLLEFRKKIAEPGSTRAAAS

SEQ ID NO: 9 - protein, 451 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSPGILGSEYEDGRGFGIGELVWGKLRGFSWWPGRIVSWWMTGRSRAAEGTRWVMWFGDGKFSVVCVEKLMPLSSFCSAFHQATYNKQPMYRKAIYEVLQVASSRAGKLFPACHDSDESDSGKAVEVQNKQMIEWALGGFQPSGPKGLEPPLERPHRDVFEVEKILDMKTEGGKVLYKVRWKGYTSDDDTWEPEIHLEDCKEVLLEFRKKIAEPGSTRAAAS

SEQ ID NO: 10 - protein, 445 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSPGILGSEYEDGRGFGIGELVWGKLRGFSWWPGRIVSWWMTGRSRAAEGTRWVMWFGDGKFSVVCVEKLMPLSSFCSAFHQATYNKQPMYRKAIYEVLQVASSRAGKLFPACHDSDESDSGKAVEVQNKQMIEWALGGFQPSGPKGLEPPDVFEVEKILDMKTEGGKVLYKVRWKGYTSDDDTWEPEIHLEDCKEVLLEFRKKIAEPGSTRAAAS

SEQ ID NO: 11 - protein, 456 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSPGILGSEYEDGRGFGIGELVWGKLRGFSWWPGRIVSWWMTGRSRAAEGTRWVMWFGDGKFSVVCVEKLMPLSSFCSAFHQATYNKQPMYRKAIYEVLQVASSRAGKLFPACHDSDESDSGKAVEVQNKQMIEWALGGFQPSGPKGLEPPLERPHRGGGGSDVFEVEKILDMKTEGGKVLYKVRWKGYTSDDDTWEPEIHLEDCKEVLLEFRKKIAEPGSTRAAAS

SEQ ID NO: 12 - protein, 223 aa, synthetic construct
MGSPGILGSEYEDGRGFGIGELVWGKLRGFSWWPGRIVSWWMTGRSRAAEGTRWVMWFGDGKFSVVCVEKLMPLSSFCSAFHQATYNKQPMYRKAIYEVLQVASSRAGKLFPACHDSDESDSGKAVEVQNKQMIEWALGGFQPSGPKGLEPPLERPHRDVFEVEKILDMKTEGGKVLYKVRWKGYTSDDDTWEPEIHLEDCKEVLLEFRKKIAEPGSTRAAAS

SEQ ID NO: 13 - protein, 217 aa, synthetic construct
MGSPGILGSEYEDGRGFGIGELVWGKLRGFSWWPGRIVSWWMTGRSRAAEGTRWVMWFGDGKFSVVCVEKLMPLSSFCSAFHQATYNKQPMYRKAIYEVLQVASSRAGKLFPACHDSDESDSGKAVEVQNKQMIEWALGGFQPSGPKGLEPPDVFEVEKILDMKTEGGKVLYKVRWKGYTSDDDTWEPEIHLEDCKEVLLEFRKKIAEPGSTRAAAS

SEQ ID NO: 14 - protein, 228 aa, synthetic construct
MGSPGILGSEYEDGRGFGIGELVWGKLRGFSWWPGRIVSWWMTGRSRAAEGTRWVMWFGDGKFSVVCVEKLMPLSSFCSAFHQATYNKQPMYRKAIYEVLQVASSRAGKLFPACHDSDESDSGKAVEVQNKQMIEWALGGFQPSGPKGLEPPLERPHRGGGGSDVFEVEKILDMKTEGGKVLYKVRWKGYTSDDDTWEPEIHLEDCKEVLLEFRKKIAEPGSTRAAAS

SEQ ID NO: 15 - protein, 596 aa, synthetic construct
MKHHHHHHHGAAGTSLYKKAGMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGENLYFQGSAHYPTRLTTRKTYSWVGRPLLDRKLHYQTYREMCVKTEGCSTEIHIQIGQFVLIEGDDDENPYVAKLLELFEDDSDPPPKKRARVQWFVRFCEVPACKRHLLGRKPGAQEIFWYDYPACDSNINAETIIGLVRVIPLAPKDVVPTNLKNEKTLFVKLSWNEKKFRPLSSELFAELNKPQESAAK

SEQ ID NO: 16 - protein, 790 aa, synthetic construct
MKHHHHHHHGAAGTSLYKKAGMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGENLYFQGSAHYPTRLTTRKTYSWVGRPLLDRKLHYQTYREMCVKTEGCSTEIHIQIGQFVLIEGDDDENPYVAKLLELFEDDSDPPPKKRARVQWFVRFCEVPACKRHLLGRKPGAQEIFWYDYPACDSNINAETIIGLVRVIPLAPKDVVPTNLKNEKTLFVKLSWNEKKFRPLSSELFAELNKPQESAAKGGGGSGGGGSAHYPTRLTTRKTYSWVGRPLLDRKLHYQTYREMCVKTEGCSTEIHIQIGQFVLIEGDDDENPYVAKLLELFEDDSDPPPKKRARVQWFVRFCEVPACKRHLLGRKPGAQEIFWYDYPACDSNINAETIIGLVRVIPLAPKDVVPTNLKNEKTLFVKLSWNEKKFRPLSSELFAELNKPQESAAK

SEQ ID NO: 17 - protein, 984 aa, synthetic construct
MKHHHHHHHGAAGTSLYKKAGMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGENLYFQGSAHYPTRLTTRKTYSWVGRPLLDRKLHYQTYREMCVKTEGCSTEIHIQIGQFVLIEGDDDENPYVAKLLELFEDDSDPPPKKRARVQWFVRFCEVPACKRHLLGRKPGAQEIFWYDYPACDSNINAETIIGLVRVIPLAPKDVVPTNLKNEKTLFVKLSWNEKKFRPLSSELFAELNKPQESAAKGGGGSGGGGSAHYPTRLTTRKTYSWVGRPLLDRKLHYQTYREMCVKTEGCSTEIHIQIGQFVLIEGDDDENPYVAKLLELFEDDSDPPPKKRARVQWFVRFCEVPACKRHLLGRKPGAQEIFWYDYPACDSNINAETIIGLVRVIPLAPKDVVPTNLKNEKTLFVKLSWNEKKFRPLSSELFAELNKPQESAAKGGGGSGGGGSAHYPTRLTTRKTYSWVGRPLLDRKLHYQTYREMCVKTEGCSTEIHIQIGQFVLIEGDDDENPYVAKLLELFEDDSDPPPKKRARVQWFVRFCEVPACKRHLLGRKPGAQEIFWYDYPACDSNINAETIIGLVRVIPLAPKDVVPTNLKNEKTLFVKLSWNEKKFRPLSSELFAELNKPQESAAK

SEQ ID NO: 18 - protein, 406 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSIEENLYFQSMAPPGMRLRSGRSTGAPLTRGSGGGGSMAPPGMRLRSGRSTGAPLTRGSGGGGSGGGGSLPATGGASASPKQRRSIIRDRGPMYDDPTLPEGWTRKLKQRKSGRSAGKYDVYLINPQGKAFRSKVELIAYFEKVGDTSLDPNDFDFTVTGRGSPSRREGGHHHHHH

SEQ ID NO: 19 - protein, 406 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSIEENLYFQSMAPPGMRLRSGRSTGAPLTRGSGGGGSMAPPGMRLRSGRSTGAPLTRGSGGGGSGGGGSLPATGGASASPKQRRSIIRDRGPMYDDPTLPEGWTRKLKQRKSGRSAGKYDVFLINPQGKAFRSKVELIAYFEKVGDTSLDPNDFDFTVTGRGSPSRREGGHHHHHH

SEQ ID NO: 20 - protein, 338 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSENLYFQGGASASPKQRRSIIRDRGPMYDDPTLPEGWTRKLKQRKSGRSAGKYDVYLINPQGKAFRSKVELIAYFEKVGDTSLDPNDFDFSVTGRGSPSRREHHHHHH

SEQ ID NO: 21 - protein, 338 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSENLYFQGGASASPKQRRSIIRDRGPMYDDPTLPEGWTRKLKQRKSGRSAGKYDVFLINPQGKAFRSKVELIAYFEKVGDTSLDPNDFDFSVTGRGSPSRREHHHHHH

SEQ ID NO: 22 - protein, 390 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSIEENLYFQSMAPPGMRLRSGRSTGAPLTRGSGGGGSMAPPGMRLRSGRSTGAPLTRGSASASPKQRRSIIRDRGPMYDDPTLPEGWTRKLKQRKSGRSAGKYDVYLINPQGKAFRSKVELIAYFEKVGDTSLDPNDFDFTVTGRGSPSRREGGHHHHHH

SEQ ID NO: 23 - protein, 401 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSIEENLYFQSMAPPGMRLRSGRSTGAPLTRGSGGGGSMAPPGMRLRSGRSTGAPLTRGSGGGGSLPATGGASASPKQRRSIIRDRGPMYDDPTLPEGWTRKLKQRKSGRSAGKYDVYLINPQGKAFRSKVELIAYFEKVGDTSLDPNDFDFTVTGRGSPSRREGGHHHHHH

SEQ ID NO: 24 - protein, 411 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSIEENLYFQSMAPPGMRLRSGRSTGAPLTRGSGGGGSMAPPGMRLRSGRSTGAPLTRGSGGGGSGGGGSGGGGSLPATGGASASPKQRRSIIRDRGPMYDDPTLPEGWTRKLKQRKSGRSAGKYDVYLINPQGKAFRSKVELIAYFEKVGDTSLDPNDFDFTVTGRGSPSRREGGHHHHHH

SEQ ID NO: 25 - protein, 409 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSIEENLYFQSMAPPGMRLRSGRSTGAPLTRGSGGGGSMAPPGMRLRSGRSTGAPLTRGSGSAGSAAGSGEFKLPATGGASASPKQRRSIIRDRGPMYDDPTLPEGWTRKLKQRKSGRSAGKYDVYLINPQGKAFRSKVELIAYFEKVGDTSLDPNDFDFTVTGRGSPSRREGGHHHHHH

SEQ ID NO: 26 - protein, 415 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSIEENLYFQSMAPPGMRLRSGRSTGAPLTRGSGGGGSMAPPGMRLRSGRSTGAPLTRGSKESGSVSSEQLAQFRSLDKLPATGGASASPKQRRSIIRDRGPMYDDPTLPEGWTRKLKQRKSGRSAGKYDVYLINPQGKAFRSKVELIAYFEKVGDTSLDPNDFDFTVTGRGSPSRREGGHHHHHH

SEQ ID NO: 27 - protein, 411 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSIEENLYFQSMAPPGMRLRSGRSTGAPLTRGSGGGGSMAPPGMRLRSGRSTGAPLTRGSEGKSSGSGSESKSTKLPATGGASASPKQRRSIIRDRGPMYDDPTLPEGWTRKLKQRKSGRSAGKYDVYLINPQGKAFRSKVELIAYFEKVGDTSLDPNDFDFTVTGRGSPSRREGGHHHHHH

SEQ ID NO: 28 - protein, 415 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSIEENLYFQSGSGGSGSGGMAPPGMRLRSGRSTGAPLTRGSGGGGSMAPPGMRLRSGRSTGAPLTRGSGGGGSGGGGSLPATGGASASPKQRRSIIRDRGPMYDDPTLPEGWTRKLKQRKSGRSAGKYDVYLINPQGKAFRSKVELIAYFEKVGDTSLDPNDFDFTVTGRGSPSRREGGHHHHHH

SEQ ID NO: 29 - protein, 361 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSIEENLYFQSGSGGSGSGGKSTHPMVTRSKASASPKQRRSIIRDRGPMYDDPTLPEGWTRKLKQRKSGRSAGKYDVYLINPQGKAFRSKVELIAYFEKVGDTSLDPNDFDFTVTGRGSPSRREGGHHHHHH

SEQ ID NO: 30 - protein, 377 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSIEENLYFQSGSGGSGSGGKSTHPMVTRSKGGGGSGGGGSLPATGGASASPKQRRSIIRDRGPMYDDPTLPEGWTRKLKQRKSGRSAGKYDVYLINPQGKAFRSKVELIAYFEKVGDTSLDPNDFDFTVTGRGSPSRREGGHHHHHH

SEQ ID NO: 31 - protein, 396 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSIEENLYFQSGSGGSGSGGEELRRRLEELRRRRGGGGMRLRSGRSTGGGGLEEEIRRLEEEIRREASASPKQRRSIIRDRGPMYDDPTLPEGWTRKLKQRKSGRSAGKYDVYLINPQGKAFRSKVELIAYFEKVGDTSLDPNDFDFTVTGRGSPSRREGGHHHHHH

SEQ ID NO: 32 - protein, 390 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSIEENLYFQSGSGGSGSGGEELRRRLEELRRRRGMRLRSGRSTGLEEEIRRLEEEIRREASASPKQRRSIIRDRGPMYDDPTLPEGWTRKLKQRKSGRSAGKYDVYLINPQGKAFRSKVELIAYFEKVGDTSLDPNDFDFTVTGRGSPSRREGGHHHHHH

SEQ ID NO: 33 - protein, 412 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSIEENLYFQSGSGGSGSGGEELRRRLEELRRRRGGGGMRLRSGRSTGGGGLEEEIRRLEEEIRREGGGGSGGGGSLPATGGASASPKQRRSIIRDRGPMYDDPTLPEGWTRKLKQRKSGRSAGKYDVYLINPQGKAFRSKVELIAYFEKVGDTSLDPNDFDFTVTGRGSPSRREGGHHHHHH

SEQ ID NO: 34 - protein, 406 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSIEENLYFQSGSGGSGSGGEELRRRLEELRRRRGMRLRSGRSTGLEEEIRRLEEEIRREGGGGSGGGGSLPATGGASASPKQRRSIIRDRGPMYDDPTLPEGWTRKLKQRKSGRSAGKYDVYLINPQGKAFRSKVELIAYFEKVGDTSLDPNDFDFTVTGRGSPSRREGGHHHHHH

SEQ ID NO: 35 - protein, 609 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSENLYFQGGDTKLYCICKTPYDESKFYIGCDRCQNWYHGRCVGILQSEAELIDEYVCPQCQSTEDAMTVGGGGSMSKGEELFTGVVPILVELDGDVNGHKFSVSGEGEGDATYGKLTLKFICTTGKLPVPWPTLVTTFTYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKGGGGSEQVFAVESIRKKRVRKGKVEYLVKWKGWPPKYSTWEPEEHILDPRLVMAYEEKEERDRASGY

SEQ ID NO: 36 - protein, 326 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSENLYFQGGVIRDEWGNQIWICPGCNKPDDGSPMIGCDDCDDWYHWPCVGIMTAPPEEMQWFCPKCANKKKDKKHKKRKHRAHGGGGSDYKDDDDK

SEQ ID NO: 37 - protein, 410 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSENLYFQGGVIRDEWGNQIWICPGCNKPDDGSPMIGCDDCDDWYHWPCVGIMTAPPEEMQWFCPKCANKKKDKKHKKRKHRAHGGGGSGGGGSVIRDEWGNQIWICPGCNKPDDGSPMIGCDDCDDWYHWPCVGIMTAPPEEMQWFCPKCANKKKDKKHKKRKHRAHGGGGSDYKDDDDK

SEQ ID NO: 38 - protein, 494 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSENLYFQGGVIRDEWGNQIWICPGCNKPDDGSPMIGCDDCDDWYHWPCVGIMTAPPEEMQWFCPKCANKKKDKKHKKRKHRAHGGGGSGGGGSVIRDEWGNQIWICPGCNKPDDGSPMIGCDDCDDWYHWPCVGIMTAPPEEMQWFCPKCANKKKDKKHKKRKHRAHGGGGSGGGGSVIRDEWGNQIWICPGCNKPDDGSPMIGCDDCDDWYHWPCVGIMTAPPEEMQWFCPKCANKKKDKKHKKRKHRAHGGGGSDYKDDDDK

SEQ ID NO: 39 - protein, 410 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSENLYFQGGVIRDEWGNQIWICPGCNKPDDGSPMIGCDDCDDWYHWPCVGIMTAPPEEMQWFCPKCANKKKDKKHKKRKHRAHSVVTETVSTYVIRDEWGNQIWICPGCNKPDDGSPMIGCDDCDDWYHWPCVGIMTAPPEEMQWFCPKCANKKKDKKHKKRKHRAHGGGGSDYKDDDDK

SEQ ID NO: 40 - protein, 494 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSENLYFQGGVIRDEWGNQIWICPGCNKPDDGSPMIGCDDCDDWYHWPCVGIMTAPPEEMQWFCPKCANKKKDKKHKKRKHRAHSVVTETVSTYVIRDEWGNQIWICPGCNKPDDGSPMIGCDDCDDWYHWPCVGIMTAPPEEMQWFCPKCANKKKDKKHKKRKHRAHSVVTETVSTYVIRDEWGNQIWICPGCNKPDDGSPMIGCDDCDDWYHWPCVGIMTAPPEEMQWFCPKCANKKKDKKHKKRKHRAHGGGGSDYKDDDDK

SEQ ID NO: 41 - protein, 455 aa, synthetic construct
MGSSHHHHHHGTKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGTDYDIPTTENLYFQGHMEEEEEEYVVEKVLDRRVVKGKVEYLLKWKGFSDEDNTWEPEENLDCPDLIAEFLQSQKT

SEQ ID NO: 42 - protein, 524 aa, synthetic construct
MGSSHHHHHHGTKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGTDYDIPTTENLYFQGHMEEEEEEYVVEKVLDRRVVKGKVEYLLKWKGFSDEDNTWEPEENLDCPDLIAEFLQSQKTGGGGSGGGGSEEEEEEYVVEKVLDRRVVKGKVEYLLKWKGFSDEDNTWEPEENLDCPDLIAEFLQSQKT

SEQ ID NO: 43 - protein, 593 aa, synthetic construct
MGSSHHHHHHGTKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGTDYDIPTTENLYFQGHMEEEEEEYVVEKVLDRRVVKGKVEYLLKWKGFSDEDNTWEPEENLDCPDLIAEFLQSQKTGGGGSGGGGSEEEEEEYVVEKVLDRRVVKGKVEYLLKWKGFSDEDNTWEPEENLDCPDLIAEFLQSQKTGGGGSGGGGSEEEEEEYVVEKVLDRRVVKGKVEYLLKWKGFSDEDNTWEPEENLDCPDLIAEFLQSQKT

SEQ ID NO: 44 - protein, 454 aa, synthetic construct
MGSSHHHHHHGTKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGTDYDIPTTENLYFQGHMEEEEEEYVVEKVLDRRVVKGKVEYLLKWAGFSDEDNTWEPEENLFCPDLIAEFLQSQK

SEQ ID NO: 45 - protein, 524 aa, synthetic construct
MGSSHHHHHHGTKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGTDYDIPTTENLYFQGHMEEEEEEYVVEKVLDRRVVKGKVEYLLKWAGFSDEDNTWEPEENLFCPDLIAEFLQSQKTGGGGSGGGGSEEEEEEYVVEKVLDRRVVKGKVEYLLKWAGFSDEDNTWEPEENLFCPDLIAEFLQSQKT

SEQ ID NO: 46 - protein, 593 aa, synthetic construct
MGSSHHHHHHGTKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGTDYDIPTTENLYFQGHMEEEEEEYVVEKVLDRRVVKGKVEYLLKWAGFSDEDNTWEPEENLFCPDLIAEFLQSQKTGGGGSGGGGSEEEEEEYVVEKVLDRRVVKGKVEYLLKWAGFSDEDNTWEPEENLFCPDLIAEFLQSQKTGGGGSGGGGSEEEEEEYVVEKVLDRRVVKGKVEYLLKWAGFSDEDNTWEPEENLFCPDLIAEFLQSQKT

SEQ ID NO: 47 - protein, 407 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSPGIPDTKLYCICKTPYDESKFYIGCDRCQNWYHGRCVGILQSEAELIDEYVCPQCQSTEDAMTVLTPLTEKDYEGLKRVLRSLQAHKMAWPFLEPVDPNDAPDYYGVIKEPMDLATMEERVQRRYYEKLTEFVADMTKIFDNCRYYNPSDSPFYQCAEVLESFFVQKLKGFKASRS